(19) United States
(12) Patent Application Publication
(10) Pub. No.: US 2025/0354119 A1
(43) Pub. Date: Nov. 20, 2025

(54) METHODS FOR ENHANCING EFFICACY OF THERAPEUTIC IMMUNE CELLS

(71) Applicant: National University of Singapore, Singapore (SG)

(72) Inventors: Dario Campana, Singapore (SG); Takahiro Kamiya, Singapore (SG)

(21) Appl. No.: 19/285,735

(22) Filed: Jul. 30, 2025

Related U.S. Application Data
(63) Continuation of application No. 16/943,400, filed on Jul. 30, 2020, now Pat. No. 12,404,491.
Application No. 16/943,400 is a continuation of application No. 15/548,577, filed on Aug. 3, 2017, now Pat. No. 10,765,699.
(60) Provisional application No. 62/112,765, filed on Feb. 6, 2015.
(60) Provisional application No. 62/130,970, filed on Mar. 10, 2015.

(51) Int. Cl. C12N 5/0783; A61K 40/11; A61K 40/15; A61K 40/31; A61K 40/42; C07K 16/28

(52) U.S. Cl. CPC C12N 5/0646; A61K 40/11; A61K 40/15; A61K 40/31; A61K 40/4211; A61K 40/4224; C07K 16/2809; C07K 16/2833; C07K 16/289; C12N 5/0636; A61K 2239/48; C07K 2319/03; C12N 2510/00

(57) ABSTRACT

The present invention relates to a method of using a receptor (e.g., chimeric antigen receptor—CAR) that activates an immune response upon binding a cancer cell ligand in conjunction with a target-binding molecule that targets a protein or molecule for removal or neutralization to generate enhanced anti-cancer immune cells. The present invention also relates to engineered immune cells having enhanced therapeutic efficacy and uses thereof.

### CROSS-REFERENCE TO RELATED APPLICATIONS

This application is a continuation of U.S. patent application Ser. No. 16/943,400, filed Jul. 30, 2020, which is a continuation of U.S. patent application Ser. No. 15/548,577, filed Aug. 3, 2017, now U.S. Pat. No. 10,765,699, which is a 371 U.S. National Phase Application of International Application No. PCT/SG2016/050063, filed Feb. 5, 2016, which claims benefit to U.S. Provisional Application No. 62/112,765, filed Feb. 6, 2015, and U.S. Provisional Application No. 62/130,970, filed Mar. 10, 2015, the disclosures of which are incorporated herein by reference in their entirety for all purposes.

### REFERENCE TO A SEQUENCE LISTING

The instant application contains a Sequence Listing which has been submitted electronically in XML format and is hereby incorporated by reference in its entirety. Said XML copy, created on Jul. 5, 2023, is named 62190_701_304_SL.xml and is 87,904 bytes in size.

### BACKGROUND OF THE INVENTION

Immune cells can be potent and specific “living drugs”. Immune cells have the potential to target tumor cells while sparing normal tissues; several clinical observations indicate that they can have major anti-cancer activity. Thus, in patients receiving allogeneic hematopoietic stem cell transplantation (HSCT), T-cell-mediated graft-versus-host disease (GvHD) (Weiden, P L et al., N. Engl. J. Med. 1979;300(19):1068-1073; Appelbaum, F R Nature, 2001;411(6835):385-389; Porter, D L et al., N. Engl. J. Med. 1994;330(2):100-106; Kolb, H J et al. Blood. 1995;86(5):2041-2050; Slavin, S. et al., Blood. 1996;87(6):2195-2204), and donor natural killer (NK) cell alloreactivity (Ruggeri L, et al. Science. 2002;295(5562):2097-2100; Giebel S, et al. Blood. 2003;102(3):814-819; Cooley S, et al. Blood. 2010;116 (14): 2411-2419) are inversely related to leukemia recurrence. Besides the HSCT context, administration of antibodies that release T cells from inhibitory signals (Sharma P, et al., Nat Rev Cancer. 2011;11(11):805-812.; Pardoll D M., Nat Rev Cancer. 2012;12(4):252-264), or bridge them to tumor cells (Topp M S, et al. J. Clin. Oncol. 2011;29(18):2493-2498) produced major clinical responses in patients with either solid tumors or leukemia. Finally, infusion of genetically-modified autologous T lymphocytes induced complete and durable remission in patients with refractory leukemia and lymphoma (Maude S L, et al. N Engl J Med. 2014;371(16):1507-1517).

Nevertheless, there is a significant need for improving immune cell therapy by broadening its applicability and enhancing its efficacy.

### SUMMARY OF THE INVENTION

The present invention relates to engineered immune cells having enhanced therapeutic efficacy for, e.g., cancer therapy. In certain embodiments, the present invention provides an engineered immune cell that comprises a nucleic acid comprising a nucleotide sequence encoding an immune activating receptor, and a nucleic acid comprising a nucleotide sequence encoding a target-binding molecule linked to a localizing domain.

In other embodiments, the present invention provides the use of an engineered immune cell that comprises a gene encoding an immune activating receptor, and a gene encoding a target-binding molecule linked to a localizing domain for treating cancer, comprising administering a therapeutic amount of the engineered immune cell to a subject in need thereof.

In various embodiments, the present invention also provides a method for producing an engineered immune cell, the method comprising introducing into an immune cell a nucleic acid comprising a nucleotide sequence encoding an immune activating receptor, and a nucleic acid comprising a nucleotide sequence encoding a target-binding molecule linked to a localizing domain, thereby producing an engineered immune cell.

In some embodiments, the engineered immune cells possess enhanced therapeutic efficacy as a result of one or more of reduced graft-versus-host disease (GvHD) in a host, reduced or elimination of rejection by a host, extended survival in a host, reduced inhibition by the tumor in a host, reduced self-killing in a host, reduced inflammatory cascade in a host, or sustained natural/artificial receptor-mediated (e.g., CAR-mediated) signal transduction in a host.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1A-1B is a schematic representation of a strategy employed in the present invention. FIG. 1A is an overall mechanism of CAR mediated killing of cancer cells. FIG. 1B shows the combined expression of CAR with different formats of compartment-directed scFv (an example of a target-binding molecule linked to a localizing domain) and examples of possible targets. The CAR can be replaced by other receptors that can enhance immune cell capacity.

FIG. 2 is a schematic diagram of constructs containing scFv together with domains that localize them to specific cellular compartments. Abbreviations: B2M, B-2 microglobulin; SP, signal peptide; VL, variable light chain; VH, variable heavy chain; TM, transmembrane domain; HA, human influenza hemagglutinin. Additional constructs not listed in the figure include membrane-bound (mb) myc EEKKMP, mb myc KKTN, mb myc YQRL, mb TGN38 cytoplasmic domain, mb myc RNIKCD, linker (20-amino acid) mb EEKKMP, as well as variants of constructs without signaling peptide and with a varying number of amino acids in the CD8 transmembrane domain. The nucleotide sequence of the 10-amino acid linker is GGTGGTGGCGGCAGTGGTGGCGGTGGCTCA (SEQ ID NO: 61); the amino acid sequence is GGGGSGGGGS (SEQ ID NO: 62). The nucleotide sequence of the 20-amino acid linker is GGTGGTGGCGGCAGTGGTGGCGGTGGCTCAGGCGGTGGTGGCTCCGGTGGCGGT GGCTCT (SEQ ID NO: 63); the amino acid sequence is GGGGSGGGGSGGGGGGGGS (SEQ ID NO: 41). Various localization domains are indicated under the heading “Localization domains,” and depicts linkers in some examples, as indicated. The constructs “myc KDEL” and “PEST KDEL” show the use of more than one localization domains in a single construct.

FIGS. 3A-3C show downregulation of CD3/TCR in T cells by scFv targeting of CD3ε. FIG. 3A shows expression of surface CD3ε in Jurkat cells, transduced with either a retroviral vector containing green fluorescent protein (GFP) only (“mock”) or a vector containing GFP plus different constructs as indicated Expression of CD3ε on the cell membrane was compared to that of mock-transduced cells 1 week after transduction using an anti-CD3 antibody conjugated to allophycocyanin (BD Biosciences). All comparisons were performed after gating on GFP-positive cells. FIG. 3B depicts similar experiments performed with peripheral blood T lymphocytes expanded with anti-CD3/CD28 beads (Lifesciences). Staining was performed 1 week after transduction. FIG. 3C shows flow cytometry plots illustrating downregulation of membrane CD3ε in Jurkat cells after transduction with the constructs indicated. Dashed rectangles on the upper right quadrant of each plot enclose GFP+ CD3+ cells.

FIG. 4 shows downregulation of CD3ε and TCRαβ on the cell membrane in Jurkat T cells upon transduction with anti-CD3ε scFv-KDEL or -PEST, or -mb EEKKMP. Membrane marker expression was measured 1 week after transduction using an anti-CD3 antibody conjugated to allophycocyanin (BD Biosciences) or an anti-TCRαβ conjugated to phycoerythrin (Biolegend). Lines labeled “Control” represent labelling of mock-transduced cells. Dashed vertical line represents the upper limit of staining obtained with an isotype-matched non-reactive antibody.

FIG. 5 shows that anti-scFv and CAR can be expressed simultaneously. Flow cytometric dot plots represent staining of Jurkat cells (top row) or peripheral blood lymphocytes (bottom row) with anti-CD3 allophycocyanin antibody and goat-anti-mouse Fab2 biotin plus streptavidin conjugated to phycoerythrin (to detect the CAR). Cells were transduced with the anti-CD3 scFv-myc KDEL construct, the anti-CD19-4-1BB-CD3ζ construct, or both. After gating on GFP-positive cells, those transduced with anti-CD3 scFv-myc KDEL downregulated CD3 (left column, bottom left quadrants) and those transduced with the anti-CD19-4-1BB-CD3ζ construct expressed the CAR (middle column, top right quadrants). A substantial proportion of cells transduced with both constructs were CD3-negative and CAR-positive (right column, top left quadrants).

FIG. 6 illustrates that anti-CD19 CAR triggers T cell activation and degranulation regardless of CD3/TCR downregulation. Jurkat cells were transduced with the anti-CD3 scFv-myc KDEL construct, the anti-CD19-4-1BB-CD3ζ construct, or both. T cell activation and degranulation was compared to that of mock-transduced cells. Cells were co-cultured alone or with the CD19+ leukemia cell line OP-1 at a 1:1 ratio. After 18 hours, expression of CD69 and CD25 was tested by flow cytometry using specific antibodies (from BD Biosciences); expression of CD107a was tested after 6 hours (antibody from BD Biosciences). In the presence of OP-1 cells, CD69 and CD25 expression in CAR-expressing cells occurred regardless of whether cells were also transduced with anti-CD3 scFv-KDEL; no activation occurred in mock-or anti-CD3 scFv-myc KDEL transduced cells, or in the absence of OP-1 cells. CAR stimulation enhanced CD107 expression which was not affected by CD3/TCR downregulation.

FIG. 7 shows that anti-CD19 CAR expressed in T cells causes T cell proliferation regardless of CD3/TCR downregulation. Peripheral blood T lymphocytes were transduced with both the anti-CD3 scFv-myc KDEL construct and the anti-CD19-4-1BB-CD3ζ construct. Transduced T lymphocytes were co-cultured with OP-1 cells treated with Streck (Omaha, NE) to inhibit their proliferation for the time indicated. Expansion of CD3-positive and CD3-negative T lymphocytes expressing the anti-CD19 CAR was compared to that of mock-transduced T cells. Each symbol shows the average cell count of two parallel cultures. CAR T cell expanded equally well regardless of CD3/TCR expression.

FIG. 8 shows expression of CD7 on the membrane of peripheral blood T lymphocytes transduced with either a retroviral vector containing GFP only (“mock”) or a vector containing GFP plus and anti-CD7 scFv-myc KDEL construct. Expression of CD7 on the cell membrane was compared to that of mock-transduced cells 1 week after transduction using an anti-CD7 antibody conjugated to phycoerythrin (BD Biosciences). Dashed rectangles on the upper right quadrant of each plot enclose GFP+ CD7+ cells.

FIG. 9 depicts the downregulation of HLA Class I in T cells by scFv targeting of β2-microglubulin. Jurkat T cells were transduced with anti-β2M scFv-myc KDEL. Expression of HLA-ABC on the cell membrane was compared to that of mock-transduced cells 1 week after transduction using an anti-HLA-ABC antibody conjugated to phycoerythrin (BD Biosciences). Staining with an isotype-matched control antibody is also shown. Analysis was performed after gating on GFP-positive cells.

FIG. 10 depicts the downregulation of Killer Immunoglobulin-like Receptor (KIR) 2DL1 and KIR2DL2/DL3 in human NK cells by scFv targeting of KIR2DL1 and KIR2DL2/DL3. NK cells, expanded ex vivo and selected for KIR2DL1 expression, were transduced with anti-KIR2DL1-KIR2DL2/DL3 scFv-linker (20) AEKDEL or -EEKKMP. Expression of the corresponding KIR on the cell membrane was compared to that of mock-transduced cells 8 days after transduction using an anti-KIR2DL1 antibody conjugated to allophycocyanin (R&D Systems) or an anti-KIR2DL2/DL3 antibody conjugated to phycoerythrin (BD Biosciences). Staining with an isotype-matched control antibody is also shown. Analysis was performed after gating on GFP-positive cells.

FIG. 11 depicts the downregulation of NKG2A in human NK cells by scFv targeting. NK cells, expanded ex vivo, were transduced with anti-NKG2A scFv-EEKKMP. Expression of NKG2A on the cell membrane was compared to that of mock-transduced cells 8 days after transduction using an NKG2A antibody conjugated to phycoerythrin (Beckman Coulter). Staining with an isotype-matched control antibody is also shown. Analysis was performed after gating on GFP-positive cells.

### DETAILED DESCRIPTION OF THE INVENTION

A description of example embodiments of the invention follows.

In recent years, gains in knowledge about the molecular pathways that regulate immune cells have been paralleled by a remarkable evolution in the capacity to manipulate them ex vivo, including their expansion and genetic engineering. It is now possible to reliably prepare highly sophisticated clinical-grade immune cell products in a timely fashion. A prime example of how the anti-cancer activity of immune cells can be directed and magnified by ex vivo cell engineering is the development of chimeric antigen receptor (CAR) T cells (Eshhar, Z. et al., PNAS. 1993;90(2):720-724).

CARs are artificial multi-molecular proteins, which have been previously described (Geiger T L, et al., J Immunol. 1999;162(10):5931-5939; Brentjens R J, et al., NatMed. 2003;9(3):279-286; Cooper L J, et al., Blood. 2003;101(4):637-1644). CARs comprise an extracellular domain that binds to a specific target, a transmembrane domain, and a cytoplasmic domain. The extracellular domain and transmembrane domain can be derived from any desired source for such domains, as described in, e.g., U.S. Pat. No. 8,399,645, incorporated by reference herein in its entirety. Briefly, a CAR may be designed to contain a single-chain variable region (scFv) of an antibody that binds specifically to a target. The scFv may be linked to a T-cell receptor (TCR)-associated signaling molecule, such as CD3ζ, via transmembrane and hinge domains. Ligation of scFv to the cognate antigen triggers signal transduction. Thus, CARs can instantaneously redirect cytotoxic T lymphocytes towards cancer cells and provoke tumor cell lysis (Eshhar, Z. et al., PNAS. 1993;90(2):720-724; Geiger T L, et al., J Immunol. 1999;162(10):5931-5939; Brentjens R J, et al., NatMed. 2003;9(3):279-286; Cooper L J, et al., Blood. 2003;101(4):1637-1644; Imai C, et al., Leukemia. 2004;18:676-684). Because CD3ζ signaling alone is not sufficient to durably activate T cells (Schwartz R H. Annu Rev Immunol. 2003;21:305-334; Zang X and Allison J P. Clin Cancer Res. 2007;13(18 Pt 1):5271-5279), co-stimulatory molecules such as CD28 and 4-1BB (or CD137) have been incorporated into CAR constructs to boost signal transduction. This dual signaling design (“second generation CAR”) is useful to elicit effective anti-tumor activity from T cells (Imai C, et al., Leukemia. 2004;18:676-684; Campana D, et al., Cancer J. 2014;20(2):134-140).

A specific CAR, anti-CD19 CAR, containing both 4-1BB and CD3ζ has been described in U.S. Pat. No. 8,399,645. Infusion of autologous T cells expressing an anti-CD19-4-1BB-CD3ζ CAR resulted in dramatic clinical responses in patients with chronic lymphocytic leukemia (CLL) (Porter D L, et al., Chimeric antigen receptor-modified T cells in chronic lymphoid leukemia; 2011: N Engl J Med. 2011; 365(8):725-733; Kalos M, et al., SciTranslMed. 2011;3(95):95ra73), and acute lymphoblastic leukemia (ALL) (Grupp S A, et al., N Engl J Med. 2013;368(16):1509-1518; Maude S L, et al., N Engl J Med. 2014;371(16):1507-1517). These studies, and studies with CARs bearing different signaling modules (Till B G, et al., Blood. 2012;119(17):3940-3950; Kochenderfer J N, et al., Blood. 2012;119(12):2709-2720; Brentjens R J , et al., Blood. 2011;118(18):4817-4828; Brentjens R J, et al., Sci Transl Med. 2013;5(177):177ra138), provide a convincing demonstration of the clinical potential of this technology, and of immunotherapy in general.

The methods described herein enable rapid removal or inactivation of specific proteins in immune cells redirected by a natural or artificial receptor, e.g., CARs, thus broadening the application potential and significantly improving the function of the engineered cells. The method relies, in part, on a single construct or multiple constructs containing an immune activating receptor, e.g., a CAR (which comprises an extracellular domain (e.g., an scFv) that binds to a specific target, a transmembrane domain, and a cytoplasmic domain) together with a target-binding molecule that binds a target (e.g., protein) to be removed or neutralized; the target-binding molecule is linked to a domain (i.e., localizing domain) that directs it to specific cellular compartments, such as the Golgi or endoplasmic reticulum, the proteasome, or the cell membrane, depending on the application. For simplicity, a target-binding molecule linked to a localizing domain (LD) is sometimes referred to herein as “LD-linked target-binding molecule.”

As will be apparent from the teachings herein, a variety of immune activating receptors may be suitable for the methods of the present invention. That is, any receptor that comprises a molecule that, upon binding (ligation) to a ligand (e.g., peptide or antigen) expressed on a cancer cell, is capable of activating an immune response may be used according to the present methods. For example, as described above, the immune activating receptor can be a chimeric antigen receptor (CAR); methods for designing and manipulating a CAR is known in the art (see, Geiger T L, et al., J Immunol. 1999;162(10):5931-5939; Brentjens R J, et al., NatMed. 2003;9(3):279-286; Cooper L J, et al., Blood. 2003;101(4):1637-1644). Additionally, receptors with antibody-binding capacity can be used (e.g., CD16-4-1BB-CD3zeta receptor—Kudo K, et al. Cancer Res. 2014;74(1):93-103), which are similar to CARs, but with the scFv replaced with an antibody-binding molecule (e.g., CD16, CD64, CD32). Further, T-cell receptors comprising T-cell receptor alpha and beta chains that bind to a peptide expressed on a tumor cell in the context of the tumor cell HLA can also be used according to the present methods. In addition, other receptors bearing molecules that activate an immune response by binding a ligand expressed on a cancer cell can also be used—e.g., NKG2D-DAP10-CD3zeta receptor, which binds to NKG2D ligand expressed on tumor cells (see, e.g., Chang Y H, et al., Cancer Res. 2013; 73(6):1777-1786). All such suitable receptors collectively, as used herein, are referred to as an “immune activating receptor” or a “receptor that activates an immune response upon binding a cancer cell ligand.” Therefore, an immune activating receptor having a molecule activated by a cancer cell ligand can be expressed together with a LD-linked target-binding molecule according to the present methods.

The present methods significantly expand the potential applications of immunotherapies based on the infusion of immune cells redirected by artificial receptors. The method described is practical and can be easily incorporated in a clinical-grade cell processing. For example, a single bicistronic construct containing, e.g., a CAR and a LD-linked target-binding molecule, e.g., scFv-myc KDEL (or PEST or transmembrane) can be prepared by inserting an internal ribosomal entry site (IRES) or a 2A peptide-coding region site between the 2 cDNAs encoding the CAR and the LD-linked target-binding molecule. The design of tricistronic delivery systems to delete more than one target should also be feasible. Alternatively, separate transductions of the 2 genes (simultaneously or sequentially) could be performed. In the context of cancer cell therapy, the CAR could be replaced by an antibody-binding signaling receptor (Kudo K, et al., Cancer Res. 2014;74(1):93-103), a T-cell receptor directed against a specific HLA-peptide combination, or any receptor activated by contact with cancer cells (Chang Y H, et al., Cancer Res. 2013; 73(6):1777-1786). The results of the studies described herein with simultaneous anti-CD19-4-1BB-CD3ζ CAR and anti-CD3ε scFv-KDEL demonstrate that the signaling capacity of the CAR was not impaired.

Both the anti-CD3ε scFv-KDEL (and -PEST) tested herein stably downregulate CD3 as well as TCR expression. Residual CD3+ T cells could be removed using CD3 beads, an approach that is also available in a clinical-grade format. The capacity to generate CD3/TCR-negative cells that respond to CAR signaling represents an important advance. Clinical studies with CAR T cells have generally been performed using autologous T cells. Thus, the quality of the cell product varies from patient to patient and responses are heterogeneous. Infusion of allogeneic T cells is currently impossible as it has an unacceptably high risk of potentially fatal GvHD, due to the stimulation of the endogenous TCR by the recipient's tissue antigens. Downregulation of CD3/TCR opens the possibility of infusing allogeneic T cells because lack of endogenous TCR eliminates GvHD capacity. Allogeneic products could be prepared with the optimal cellular composition (e.g., enriched in highly cytotoxic T cells, depleted of regulatory T cells, etc.) and selected so that the cells infused have high CAR expression and functional potency. Moreover, fully standardized products could be cryopreserved and be available for use regardless of the patient immune cell status and his/her fitness to undergo apheresis or extensive blood draws. Removal of TCR expression has been addressed using gene editing tools, such as nucleases (Torikai H, et al. Blood, 2012;119(24):5697-5705). Although this is an effective approach, it is difficult to implement in a clinical setting as it requires several rounds of cell selection and expansion, with prolonged culture. The methods described herein have considerable practical advantages.

Additionally, a LD-linked target-binding molecule (e.g., scFv-myc KDEL, scFv-EEKKMP or scFv-PEST, wherein scFv targets a specific protein/molecule) can be used according to the present invention to delete HLA Class I molecules, reducing the possibility of rejection of allogeneic cells. While infusion of allogeneic T cells is a future goal of CAR T cell therapy, infusion of allogeneic natural killer (NK) cells is already in use to treat patients with cancer. A key factor that determines the success of NK cell-based therapy is that NK cells must persist in sufficient numbers to achieve an effector: target ratio likely to produce tumor cytoreduction (Miller J S. Hematology Am Soc Hematol Educ Program. 2013;2013:247-253). However, when allogeneic cells are infused, their persistence is limited. Immunosuppressive chemotherapy given to the patient allows transient engraftment of the infused NK cells but these are rejected within 2-4 weeks of infusion (Miller J S, et al. Blood. 2005;105:3051-3057; Rubnitz J E, et al., J Clin Oncol. 2010;28(6):955-959). Contrary to organ transplantation, continuing immunosuppression is not an option because immunosuppressive drugs also suppress NK cell function. Because rejection is primarily mediated by recognition of HLA Class I molecules by the recipient's CD8+ T lymphocytes, removing HLA Class I molecules from the infused NK cells (or T cells) will diminish or abrogate the rejection rate, extend the survival of allogeneic cells, and hence their anti-tumor capacity.

Furthermore, a LD-linked target-binding molecule can be used according to the present invention to target inhibitory receptors. Specifically, administration of antibodies that release T cells from inhibitory signals such as anti-PD1 or anti-CTLA-4 have produced dramatic clinical responses (Sharma P, et al., Nat Rev Cancer. 2011;11(11):805-812; Pardoll D M. Nat Rev Cancer. 2012;12(4):252-264). CAR-T cells, particularly those directed against solid tumors, might be inhibited by similar mechanisms. Thus, expression of a target-binding molecule (e.g., scFv or ligands) against PD1, CTLA-4, Tim3 or other inhibitory receptors would prevent the expression of these molecules (if linked to, e.g., KDEL (SEQ ID NO: 4), EEKKMP (SEQ ID NO: 64) or PEST motif SHGFPPEVEEQDDGTLPMSCAQESGMDRHPAACASARINV (SEQ ID NO: 7)) or prevent binding of the receptors to their ligands (if linked to a transmembrane domain) and sustain CAR-mediated signal transduction. In NK cells, examples of inhibitory receptors include killer immunoglobulin-like receptors (KIRs) and NKG2A (Vivier E, et al., Science, 2011;331(6013):44-49).

The methods of the present invention also enable targeting of a greater number of targets amenable for CAR-directed T cell therapy. One of the main limitations of CAR-directed therapy is the paucity of specific antigens expressed by tumor cells. In the case of hematologic malignancies, such as leukemias and lymphomas, molecules which are not expressed in non-hematopoietic cells could be potential targets but cannot be used as CAR targets because they are also expressed on T cells and/or NK cells. Expressing such CARs on immune cells would likely lead to the demise of the immune cells themselves by a “fratricidal” mechanism, nullifying their anti-cancer capacity. If the target molecule can be removed from immune cells without adverse functional effects, then the CAR with the corresponding specificity can be expressed. This opens many new opportunities to target hematologic malignancies. Examples of the possible targets include CD38 expressed in multiple myeloma, CD7 expressed in T cell leukemia and lymphoma, Tim-3 expressed in acute leukemia, CD30 expressed in Hodgkin disease, CD45 and CD52 expressed in all hematologic malignancies. These molecules are also expressed in a substantial proportion of T cells and NK cells.

Moreover, it has been shown that secretion of cytokines by activated immune cells triggers cytokine release syndrome and macrophage activation syndrome, presenting serious adverse effects of immune cell therapy (Lee D W, et al., Blood. 2014;124(2):188-195). Thus, the LD-linked target-binding molecule can be used according to the present invention to block cytokines such as IL-6, IL-2, IL-4, IL-7, IL-10, IL-12, IL-15, IL-18, IL-21, IL-27, IL-35, interferon (IFN)-γ, IFN-β, IFN-α, tumor necrosis factor (TNF)-α, and transforming growth factor (TGF)-β, which may contribute to such inflammatory cascade.

Accordingly, in one embodiment, the present invention relates to an engineered immune cell that comprises a nucleic acid comprising a nucleotide sequence encoding an immune activating receptor, and a nucleic acid comprising a nucleotide sequence encoding a target-binding molecule linked to a localizing domain.

As used herein, an “engineered” immune cell includes an immune cell that has been genetically modified as compared to a naturally-occurring immune cell. For example, an engineered T cell produced according to the present methods carries a nucleic acid comprising a nucleotide sequence that does not naturally occur in a T cell from which it was derived. In some embodiments, the engineered immune cell of the present invention includes a chimeric antigen receptor (CAR) and a target-binding molecule linked to a localizing domain (LD-linked target-binding molecule). In a particular embodiment, the engineered immune cell of the present invention includes an anti-CD19-4-1BB-CD3ζ CAR and an anti-CD3 scFv linked to a localizing domain.

In certain embodiments, the engineered immune cell is an engineered T cell, an engineered natural killer (NK) cell, an engineered NK/T cell, an engineered monocyte, an engineered macrophage, or an engineered dendritic cell.

In certain embodiments, an “immune activating receptor” as used herein refers to a receptor that activates an immune response upon binding a cancer cell ligand. In some embodiments, the immune activating receptor comprises a molecule that, upon binding (ligation) to a ligand (e.g., peptide or antigen) expressed on a cancer cell, is capable of activating an immune response. In one embodiment, the immune activating receptor is a chimeric antigen receptor (CAR); methods for designing and manipulating a CAR are known in the art. In other embodiments, the immune activating receptor is an antibody-binding receptor, which is similar to a CAR, but with the scFv replaced with an antibody-binding molecule (e.g., CD16, CD64, CD32) (see e.g., CD16-4-1BB-CD3zeta receptor—Kudo K, et al. Cancer Res. 2014;74(1):93-103). In various embodiments, T-cell receptors comprising T-cell receptor alpha and beta chains that bind to a peptide expressed on a tumor cell in the context of the tumor cell HLA can also be used according to the present methods. In certain embodiments, other receptors bearing molecules that activate an immune response by binding a ligand expressed on a cancer cell can also be used—e.g., NKG2D-DAP10-CD3zeta receptor, which binds to NKG2D ligand expressed on tumor cells (see, e.g., Chang Y H, et al., Cancer Res. 2013; 73(6):1777-1786). All such suitable receptors capable of activating an immune response upon binding (ligation) to a ligand (e.g., peptide or antigen) expressed on a cancer cell are collectively referred to as an “immune activating receptor.” As would be appreciated by those of skill in the art, an immune activating receptor need not contain an antibody or antigen-binding fragment (e.g., scFv); rather the portion of the immune activating receptor that binds to a target molecule can be derived from, e.g., a receptor in a receptor-ligand pair, or a ligand in a receptor-ligand pair.

In certain aspects, the immune activating receptor binds to molecules expressed on the surface of tumor cells, including but not limited to, CD20, CD22, CD33, CD2, CD3, CD4, CD5, CD7, CD8, CD45, CD52, CD38, CS-1, TIM3, CD123, mesothelin, folate receptor, HER2-neu, epidermal-growth factor receptor, and epidermal growth factor receptor. In some embodiments, the immune activating receptor is a CAR (e.g., anti-CD19-4-1BB-CD3ζ CAR). In certain embodiments, the immune activating receptor comprises an antibody or antigen-binding fragment thereof (e.g., scFv) that binds to molecules expressed on the surface of tumor cells, including but not limited to, CD20, CD22, CD33, CD2, CD3, CD4, CD5, CD7, CD8, CD45, CD52, CD38, CS-1, TIM3, CD123, mesothelin, folate receptor, HER2-neu, epidermal-growth factor receptor, and epidermal growth factor receptor. Antibodies to such molecules expressed on the surface of tumor cells are known and available in the art. By way of example, antibodies to CD3 and CD7 are commercially available and known in the art. Such antibodies, as well as fragments of antibodies (e.g., scFv) derived therefrom, can be used in the present invention, as exemplified herein. Further, methods of producing antibodies and antibody fragments against a target protein are well-known and routine in the art.

The transmembrane domain of an immune activating receptor according to the present invention (e.g., CAR) can be derived from a single-pass membrane protein, including, but not limited to, CD8α, CD8β, 4-1BB, CD28, CD34, CD4, FcεRIγ, CD16 (e.g., CD16A or CD16B), OX40, CD3ζ, CD3ε, CD3δ, CD3ε, TCRα, CD32 (e.g., CD32A or CD32B), CD64 (e.g., CD64A, CD64B, or CD64C), VEGFR2, FAS, and FGFR2B. In some examples, the membrane protein is not CD8α. The transmembrane domain may also be a non-naturally occurring hydrophobic protein segment.

The hinge domain of the immune activating receptor (e.g., CAR) can be derived from a protein such as CD8α, or IgG. The hinge domain can be a fragment of the transmembrane or hinge domain of CD8α, or a non-naturally occurring peptide, such as a polypeptide consisting of hydrophilic residues of varying length, or a (GGGGS)ₙ (SEQ ID NO: 8) polypeptide, in which n is an integer of, e.g., 3-12, inclusive.

The signaling domain of the immune activating receptor (e.g., CAR) can be derived from CD3ζ, FcεRIγ, DAP10, DAP12 or other molecules known to deliver activating signals in immune cells. At least one co-stimulatory signaling domain of the receptor can be a co-stimulatory molecule such as 4-1BB (also known as CD137), CD28, CD28_{LL→GG} variant, OX40, ICOS, CD27, GITR, HVEM, TIM1, LFA1, or CD2. Such molecules are readily available and known in the art.

As would be appreciated by those of skill in the art, the components of an immune activating receptor can be engineered to comprise a number of functional combinations, as described herein, to produce a desired result. Using the particular CAR anti-CD19-4-1BB-CD3ζ as an example, the antibody (e.g., or antigen-binding fragment thereof such as an scFv) that binds a molecule can be substituted for an antibody that binds different molecule, as described herein (e.g., anti-CD20, anti-CD33, anti-CD123, etc., instead of anti-CD19). In other embodiments, the co-stimulatory molecule (4-1BB in this specific example) can also be varied with a different co-stimulatory molecule, e.g., CD28. In some embodiments, the stimulatory molecule (CD3ζ in this specific example), can be substituted with another known stimulatory molecule. In various embodiments, the transmembrane domain of the receptor can also be varied as desired. The design, production, and testing for functionality of such immune activating receptors can be readily determined by those of skill in the art. Similarly, the design, delivery into cells and expression of nucleic acids encoding such immune activating receptors are readily known and available in the art.

As used herein, the term “nucleic acid” refers to a polymer comprising multiple nucleotide monomers (e.g., ribonucleotide monomers or deoxyribonucleotide monomers). “Nucleic acid” includes, for example, genomic DNA, cDNA, RNA, and DNA-RNA hybrid molecules. Nucleic acid molecules can be naturally occurring, recombinant, or synthetic. In addition, nucleic acid molecules can be single-stranded, double-stranded or triple-stranded. In some embodiments, nucleic acid molecules can be modified. In the case of a double-stranded polymer, “nucleic acid” can refer to either or both strands of the molecule.

The term “nucleotide sequence,” in reference to a nucleic acid, refers to a contiguous series of nucleotides that are joined by covalent linkages, such as phosphorus linkages (e.g., phosphodiester, alkyl and aryl-phosphonate, phosphorothioate, phosphotriester bonds), and/or non-phosphorus linkages (e.g., peptide and/or sulfamate bonds). In certain embodiments, the nucleotide sequence encoding, e.g., a target-binding molecule linked to a localizing domain is a heterologous sequence (e.g., a gene that is of a different species or cell type origin).

The terms “nucleotide” and “nucleotide monomer” refer to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases known in the art.

As will be appreciated by those of skill in the art, in some aspects, the nucleic acid further comprises a plasmid sequence. The plasmid sequence can include, for example, one or more sequences selected from the group consisting of a promoter sequence, a selection marker sequence, and a locus-targeting sequence.

As used herein, the gene encoding a target-binding molecule linked to a localizing domain is sometimes referred to as “LD-linked target-binding molecule.”

In certain embodiments, the target-binding molecule is an antibody or antigen-binding fragment thereof. As used herein, “antibody” means an intact antibody or antigen-binding fragment of an antibody, including an intact antibody or antigen-binding fragment that has been modified or engineered, or that is a human antibody. Examples of antibodies that have been modified or engineered are chimeric antibodies, humanized antibodies, multiparatopic antibodies (e.g., biparatopic antibodies), and multispecific antibodies (e.g., bispecific antibodies). Examples of antigen-binding fragments include Fab, Fab′, F(ab′)₂, Fv, single chain antibodies (e.g., scFv), minibodies and diabodies.

A “Fab fragment” comprises one light chain and the C_{H}1 and variable regions of one heavy chain. The heavy chain of a Fab molecule cannot form a disulfide bond with another heavy chain molecule.

An “Fc” region contains two heavy chain fragments comprising the CH2 and CH3 domains of an antibody. The two heavy chain fragments are held together by two or more disulfide bonds and by hydrophobic interactions of the CH3 domains.

A “Fab′ fragment” contains one light chain and a portion of one heavy chain that contains the VH domain and the CH1 domain and also the region between the CH1 and CH2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}² domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

In a particular embodiment, the target-binding molecule is single-chain Fv antibody (“scFv antibody”). scFv refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. For a review of scFv, see Pluckthun (1994) The Pharmacology Of Monoclonal Antibodies, vol. 113, Rosenburg and Moore eds. Springer-Verlag, New York, pp. 269-315. See also, PCT Publication No. WO 88/01649 and U.S. Pat. Nos. 4,946,778 and 5,260,203. By way of example, the linker between the VH and VL domains of the scFvs disclosed herein comprise, e.g., GGGGSGGGGSGGGGSGGGGS (SEQ ID NO: 41) or GGGGSGGGGSGGGGS (SEQ ID NO: 43). As would be appreciated by those of skill in the art, various suitable linkers can be designed and tested for optimal function, as provided in the art, and as disclosed herein.

The scFv that is part of the LD-linked target-binding molecule is not necessarily the same as the scFv that occurs in the context of, e.g., a chimeric antigen receptor (CAR) or a similar antibody-binding signaling receptor. In some embodiments, the scFv that is part of the LD-linked target-binding molecule is the same as the scFv that occurs in the context of, e.g., a chimeric antigen receptor (CAR) or a similar antibody-binding signaling receptor.

In some embodiments, the nucleic acid comprising a nucleotide sequence encoding a target-binding molecule (e.g., an scFv in the context of a LD-linked target-binding molecule) comprises one or more sequences that have at least 80%, at least 85%, at least 88%, at least 90%, at least 92%, at least 94%, at least 95%, at least 96%, at least 97%, at least 98%, at least 99%, or 100% sequence identity to any one or more of SEQ ID NOs: 14, 15, 18, 19, 22, 23, 26, 27, 30, 31, 34, 35, 38, or 39.

The term “sequence identity” means that two nucleotide or amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 95% sequence identity or more. For sequence comparison, typically one sequence acts as a reference sequence (e.g., parent sequence), to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In certain embodiments, the antibody (e.g., scFv) comprises VH and VL having amino acid sequences set forth in SEQ ID NO: 12 and 13, respectively; SEQ ID NO: 16 and 17, respectively; SEQ ID NO: 20 and 21, respectively; SEQ ID NO: 24 and 25, respectively; SEQ ID NO: 28 and 29, respectively; SEQ ID NO: 32 and 33, respectively; or SEQ ID NO: 36 and 37, respectively. In some embodiments, the antibody (e.g., scFv) comprises VH and VL having sequence that each have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NO: 12 and 13, respectively; SEQ ID NO: 16 and 17, respectively; SEQ ID NO: 20 and 21, respectively; SEQ ID NO: 24 and 25, respectively; SEQ ID NO: 28 and 29, respectively; SEQ ID NO: 32 and 33, respectively; or SEQ ID NO: 36 and 37, respectively.

A “diabody” is a small antibody fragment with two antigen-binding sites. The fragments comprise a heavy chain variable region (VH) connected to a light chain variable region (VL) in the same polypeptide chain (VH-VL or VL-VH). By using a linker that is too short to allow pairing between the two domains on the same chain, the domains are forced to pair with the complementary domains of another chain and create two antigen-binding sites. Diabodies are described in, e.g., patent documents EP 404,097; WO 93/11161; and Holliger et al., (1993) Proc. Natl. Acad. Sci. USA 90:6444-6448.

In certain embodiments, the antibody is a triabody or a tetrabody. Methods of designing and producing triabodies and tetrabodies are known in the art. See, e.g., Todorovska et al., J. Immunol. Methods 248 (1-2):47-66, 2001.

A “domain antibody fragment” is an immunologically functional immunoglobulin fragment containing only the variable region of a heavy chain or the variable region of a light chain. In some instances, two or more VH regions are covalently joined with a peptide linker to create a bivalent domain antibody fragment. The two VH regions of a bivalent domain antibody fragment may target the same or different antigens.

In some embodiments, the antibody is modified or engineered. Examples of modified or engineered antibodies include chimeric antibodies, multiparatopic antibodies (e.g., biparatopic antibodies), and multispecific antibodies (e.g., bispecific antibodies).

As used herein, “multiparatopic antibody” means an antibody that comprises at least two single domain antibodies, in which at least one single domain antibody is directed against a first antigenic determinant on an antigen and at least one other single domain antibody is directed against a second antigenic determinant on the same antigen. Thus, for example, a “biparatopic” antibody comprises at least one single domain antibody directed against a first antigenic determinant on an antigen and at least one further single domain antibody directed against a second antigenic determinant on the same antigen.

As used herein, “multispecific antibody” means an antibody that comprises at least two single domain antibodies, in which at least one single domain antibody is directed against a first antigen and at least one other single domain antibody is directed against a second antigen (different from the first antigen). Thus, for example, a “bispecific” antibody is one that comprises at least one single domain antibody directed against a first antigen and at least one further single domain antibody directed against a second antigen, e.g., different from the first antigen.

In some embodiments, the antibodies disclosed herein are monoclonal antibodies, e.g., murine monoclonal antibodies. Methods of producing monoclonal antibodies are known in the art. See, for example, Pluckthun (1994) The Pharmacology of Monoclonal Antibodies, Vol. 113, Rosenburg and Moore eds. Springer-Verlag, New York, pp. 269-315.

In various embodiments, the target-binding molecule in the context of a LD-linked target-binding molecule is a receptor or a ligand that binds to a target molecule. For example, that target-binding molecule can be a ligand that binds PD-1 (e.g., PD-L1 or PD-L2). Thus, as would be appreciated by those of skill in the art, the target-binding molecule can be an antibody, or a ligand/receptor that binds a target molecule.

As used herein, “linked” in the context of a LD-linked target-binding molecule refers to a gene encoding a target-binding molecule directly in frame (e.g., without a linker) adjacent to one or more genes encoding one or more localizing domains. Alternatively, the gene encoding a target-binding molecule may be connected to one or more gene encoding one or more localizing domains through a linker sequence, as described herein. Various suitable linkers known in the art can be used to tether the target-binding molecule to a localizing domain. For example, non-naturally occurring peptides, such as a polypeptide consisting of hydrophilic residues of varying length, or a (GGGGS)ₙ (SEQ ID NO: 8) polypeptide, in which n is an integer of, e.g., 3-12, inclusive, can be used according to the present invention. In particular embodiments, the linker comprises, e.g., GGGGSGGGGS (SEQ ID NO: 62). In some embodiments, the linker comprises, e.g., GGGGSGGGGSGGGGSGGGGS (SEQ ID NO: 41). In various embodiments, peptide linkers having lengths of about 5 to about 100 amino acids, inclusive, can be used in the present invention. In certain embodiments, peptide linkers having lengths of about 20 to about 40 amino acids, inclusive, can be used in the present invention. In some embodiments, peptide linkers having lengths of at least 5 amino acids, at least 10 amino acids, at least 15 amino acids, at least 20 amino acids, at least 25 amino acids, at least 30 amino acids, at least 35 amino acids, or at least 40 amino acids can be used in the present invention. As would be appreciated by those of skill in the art, such linker sequences as well as variants of such linker sequences are known in the art. Methods of designing constructs that incorporate linker sequences as well as methods of assessing functionality are readily available to those of skill in the art.

In certain embodiments, the LD-linked target-binding molecule binds to a target expressed on the surface of an immune cell. In some embodiments, the LD-linked target-binding molecule inhibits the activity or function of the target molecule. By way of example, as disclosed herein, the LD-linked target-binding molecule can be designed to bind to, e.g., CD3, CD7, CD45, hB2MG, KIR2DL1, KIR2DL2/DL3, or NKG2A, thereby downregulating the cell surface expression of such molecules. Downregulation of such molecules can be achieved through, for example, localizing/targeting the molecules for degradation and/or internalization. In other embodiments, the LD-linked target-binding molecule renders the target inactive (e.g., the target can no longer interact and/or bind to its cognate ligand or receptor).

In some embodiments, the engineered immune cells of the present invention have enhanced therapeutic efficacy. As used herein, “enhanced therapeutic efficacy” refers to one or more of reduced graft-versus-host disease (GvHD) in a host, reduced or elimination of rejection by a host, extended survival in a host, reduced inhibition by the tumor in a host, reduced self-killing in a host, reduced inflammatory cascade in a host, or sustained CAR-mediated signal transduction in a host.

In certain embodiments of the present invention, the target-binding molecule in the context of a LD-linked target-binding molecule binds to a molecule in a CD3/T-cell receptor (TCR) complex, a cytokine, a human leukocyte antigen (HLA) Class I molecule, or a receptor that downregulates immune response.

In certain embodiments, a molecule in a CD3/TCR complex can be CD3ε, TCRα, TCRβ, TCRγ, TCRδ, CD3ε, CD3γ, or CD3ζ. In a particular embodiment, the molecule is CD3ε.

In another embodiment, the HLA Class I molecule is beta-2 microglobulin, α1-microglobulin, α2-microglobulin, or α3-microglobulin.

In other embodiments, a receptor that downregulates immune response is selected from, e.g., PD-1, CTLA-4, Tim3, killer immunoglobulin-like receptors (KIRs—e.g., KIR2DL1 (also known as CD158a), KIR2DL2/DL3 (also known as CD158b)), CD94 or NKG2A (also known as CD159a), protein tyrosine phosphatases such as Src homology region 2 domain-containing phosphatase (SHP)-1 and SHP-2. Thus, such receptors can be targeted by moiety LD-linked target-binding molecule, as described herein.

In various embodiments, examples of cytokines that can be targeted with moiety LD-linked target-binding molecule include, e.g., interleukin (IL)-6, IL-2, IL-4, IL-7, IL-10, IL-12, IL-15, IL-18, IL-21, IL-27, IL-35, interferon (IFN)-γ, IFN-β, IFN-α, tumor necrosis factor (TNF)-α, or transforming growth factor (TGF)-β.

In a further aspect, the LD-linked target-binding molecule binds to a molecule selected from, e.g., CD2, CD4, CD5, CD7, CD8, CD30, CD38, CD45, CD52, or CD127.

Methods of producing antibodies and antibody fragments thereof against any target protein are well-known and routine in the art. Moreover, as exemplified herein, commercially available antibodies to various targets, e.g., CD3 and CD7 can be used to generate a LD-linked target-binding molecule, as exemplified herein. Antibodies known in the art, as well as fragments of antibodies (e.g., scFv) derived therefrom, can be used in the present invention, as exemplified herein.

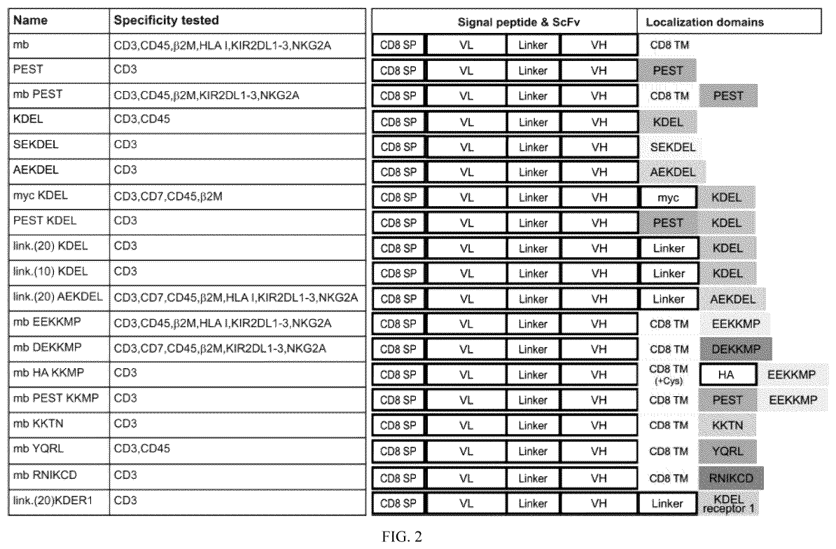

In other aspects, the localizing domain of the LD-linked target-binding molecule comprises an endoplasmic reticulum (ER) retention sequence KDEL (SEQ ID NO: 4), or other ER or Golgi retention sequences such as KKXX (SEQ ID NO: 9), KXD/E (SEQ ID NO: 10) (where X can be any amino acid—see Gao C, et al., Trends in Plant Science 19:508-515, 2014) and YQRL (SEQ ID NO: 11) (see Zhan J, et al., Cancer Immunol Immunother 46:55-60, 1998); a proteosome targeting sequence that comprises, e.g., “PEST” motif—SHGFPPEVEEQDDGTLPMSCAQESGMDRHPAACASARINV (SEQ ID NO: 7); and/or a sequence that targets the target-binding molecule to the cell membrane, such as the CD8α transmembrane domain, or the transmembrane of another single-pass membrane protein, as described herein (e.g., CD8α, CD8β, 4-1BB, CD28, CD34, CD4, FcεRIγ, CD16 (such as CD16A or CD16B), OX40, CD3ζ, CD3ε, CD3γ, CD3δ, TCRα, CD32 (such as CD32A or CD32B), CD64 (such as CD64A, CD64B, or CD64C), VEGFR2, FAS, or FGFR2B). Examples of particular localizing domains (sequences) exemplified herein are shown in FIG. 2. Various other localizing sequences are known and available in the art.

As shown in FIG. 2, the LD-linked target-binding molecules of the present invention can comprise one or more localizing domains. For example, the LD-linked target-binding molecule can have at least one, at least two, at least three, at least four, at least five, at least six, at least seven, at least eight, at least nine, or at least ten localizing domains linked together. When more than one localizing domain is used in a given LD-linked target-binding molecule, each localizing domain can be linked with or without any intervening linker. By way of example, as shown in FIG. 2, localization domains CD8 TM, PEST motif, and EEKKMP can be used in a single LD-linked target-binding molecule. While this particular construct shows the localization domains without any intervening linkers, various intervening linkers can be incorporated between some or all of the localization domains. Other examples are shown in FIG. 2.

As would be appreciated by those of skill in the art, the immune activating receptor and/or the LD-linked target-binding molecule can be designed to bind to the targets disclosed herein, as well as variants of the targets disclosed herein. By way of example, an immune activating receptor and/or the LD-linked target-binding molecule can be designed to bind to a molecule in a CD3/TCR complex, or a naturally-occurring variant molecule thereof. Such naturally-occurring variants can have the same function as the wild-type form of the molecule. In other embodiments, the variant can have a function that is altered relative to the wild-type form of the molecule (e.g., confers a diseased state).

As would be appreciated by those of skill in the art, the various components of the LD-linked target-binding molecule constructs shown in FIG. 2 can be substituted in different combinations (e.g., to contain a different linker, different localizing sequence, different scFv, etc.), so long as the combination produces a functional LD-linked target-binding molecule. Methods of assessing functionality for a particular construct are within the ambit of those of skill in the art, as disclosed herein.

In further aspects, the present invention relates to the use of an engineered immune cell that comprises a nucleic acid comprising a nucleotide sequence encoding an immune activating receptor, and a nucleic acid comprising a nucleotide sequence encoding a target-binding molecule (e.g., scFv) linked to a localizing domain for treating cancer, comprising administering a therapeutic amount of the engineered immune cell to a subject in need thereof.

In another aspect, the present invention relates to the use of an engineered immune cell that comprises a nucleic acid comprising a nucleotide sequence encoding a chimeric antigen receptor (CAR) and a nucleic acid comprising a nucleotide sequence encoding a single-chain variable fragment (scFv) linked to a localizing domain for treating cancer, comprising administering a therapeutic amount of the engineered immune cell to a subject in need thereof.

In other aspects, the present invention relates to the use of an engineered immune cell that comprises a nucleic acid comprising a nucleotide sequence encoding an immune activating receptor, and a nucleic acid comprising a nucleotide sequence encoding a target-binding molecule (e.g., scFv) linked to a localizing domain for treating an autoimmune disorder, comprising administering a therapeutic amount of the engineered immune cell to a subject in need thereof.

In other aspects, the present invention also relates to the use of an engineered immune cell that comprises a nucleic acid comprising a nucleotide sequence encoding an immune activating receptor, and a nucleic acid comprising a nucleotide sequence encoding a target-binding molecule (e.g., scFv) linked to a localizing domain for treating an infectious disease, comprising administering a therapeutic amount of the engineered immune cell to a subject in need thereof.

In various embodiments, the immune activating receptor is a CAR (e.g., anti-

CD19-4-1BB-CD3ζ CAR).

In other embodiments, the single-chain variable fragment (scFv) linked to a localizing domain is selected from any one or more constructs shown in FIG. 2.

In some aspects, the engineered immune cell is administered by infusion into the subject. Methods of infusing immune cells (e.g., allogeneic or autologous immune cells) are known in the art. A sufficient number of cells are administered to the recipient in order to ameliorate the symptoms of the disease. Typically, dosages of 10⁷ to 10¹⁰ cells are infused in a single setting, e.g., dosages of 10⁹ cells. Infusions are administered either as a single 10⁹ cell dose or divided into several 10⁹ cell dosages. The frequency of infusions can be every 3 to 30 days or even longer intervals if desired or indicated. The quantity of infusions is generally at least 1 infusion per subject and preferably at least 3 infusions, as tolerated, or until the disease symptoms have been ameliorated. The cells can be infused intravenously at a rate of 50-250 ml/hr. Other suitable modes of administration include intra-arterial infusion, direct injection into tumor and/or perfusion of tumor bed after surgery, implantation at the tumor site in an artificial scaffold, intrathecal administration, and intraocular administration. Methods of adapting the present invention to such modes of delivery are readily available to one skilled in the art.

In certain aspects, the cancer to be treated is a solid tumor or a hematologic malignancy. Examples of hematologic malignancies include acute myeloid leukemia, chronic myelogenous leukemia, myelodysplasia, acute lymphoblastic leukemia, chronic lymphocytic leukemia, multiple myeloma, Hodgkin and non-Hodgkin lymphoma. Examples of solid tumors include lung cancer, melanoma, breast cancer, prostate cancer, colon cancer, renal cell carcinoma, ovarian cancer, pancreatic cancer, hepatocellular carcinoma, neuroblastoma, rhabdomyosarcoma, brain tumor.

In another embodiment, the present invention relates to a method for producing an engineered immune cell of the present invention, comprising introducing into an immune cell a nucleic acid comprising a nucleotide sequence encoding an immune activating receptor, and a nucleic acid comprising a nucleotide sequence encoding a target-binding molecule linked to a localizing domain, thereby producing an engineered immune cell.

In certain embodiments, the nucleic acid comprising a nucleotide sequence is introduced into an immune cell ex vivo. In other embodiments, the nucleic acid comprising a nucleotide sequence is introduced into an immune cell in vivo.

In some embodiments, an “immune cell” includes, e.g., a T cell, a natural killer (NK) cell, an NK/T cell, a monocyte, a macrophage, or a dendritic cell.

The nucleic acid comprising a nucleotide sequence to be introduced can be a single bicistronic construct containing an immune activating receptor described herein and a target-binding molecule (e.g., scFv) linked to a localizing domain. As described herein, a single bicistronic construct can be prepared by inserting an internal ribosomal entry site (IRES) or a 2A peptide-coding region site between the 2 cDNAs encoding the immune activating receptor as described herein (e.g., CAR) and the target-binding molecule (e.g., scFv). The design of tricistronic delivery systems to delete more than one target should also be feasible. Alternatively, separate transductions (simultaneously or sequentially) of the individual constructs (e.g., CAR and LD-linked target-binding molecule) could be performed. Methods of introducing exogenous nucleic acids are exemplified herein, and are well-known in the art.

As used herein, the indefinite articles “a” and “an” should be understood to mean “at least one” unless clearly indicated to the contrary.

### EXEMPLIFICATION

### Methods

Cloning of scFv From Mouse Anti-Human CD3 Hybridoma

PLU4 hybridoma cells secreting an anti-human CD3 monoclonal antibody (IgG2a isotype; Creative Diagnostics, Shirley, NY) were cultured in IMDM plus GlutaMAX medium (Life Technologies, Carlsbad, CA) with 20% fetal bovine serum (Thermo Fisher Scientific, Waltham, MA) and antibiotics. Total RNA was extracted using TRIzol reagent (Life Technologies), and cDNA was synthesized by M-MLV reverse transcriptase (Promega, Madison, WI) and Oligo(dT)₁₅ primer (Promega). IgG Library Primer Set Mouse BioGenomics (US Biological, Salem, MA) was used to amplify the variable region of heavy chain (VH) and light chain (VL); PCR products were cloned into TOPO TA cloning kit for sequencing (Life Technologies). The VH and VL genes were assembled into scFv by a flexible linker sequence which encodes (Gly₄Ser)₄ using splicing by overlapping extension-PCR. Signal peptide domain of CD8α was subcloned by PCR using cDNA derived from human activated T cell from healthy donor, and connected to 5′ end of the VL fragment. The Myc tag (EQKLISEEDL; SEQ ID NO: 1) was added to C-terminus of VH by PCR using sense primer: 5′—ATATATGAATTCGGCTTCCACCATGGCCTTACCAGTGACC-3′ (SEQ ID NO: 2) and reverse primer: 5′—CAGATCTTCTTCAGAAATAAGTTTTTGTTCGGCTGAGGAGACTGTGAGAG-3′ (SEQ ID NO: 3). Also the KDEL (SEQ ID NO: 4) coding sequence was generated after Myc tag by sense primer: 5′—ATATATGAATTCGGCTTCCACCATGGCCTTACCAGTGACC-3′ (SEQ ID NO: 5) and reverse primer: 5′-TATATACTCGAGTTACAACTCGTCCTTCAGATCTTCTTCAGAAATAAG-3′ (SEQ ID NO: 6). The synthesized gene consisting of CD8 signal peptide, scFv against human CD3, Myc tag and KDEL (SEQ ID NO: 4) sequence was subcloned into EcoRI and XhoI sites of the MSCV-IRES-GFP vector. Constructs in which myc-KDEL was replaced by other sequences were also made as listed in FIG. 2.

The sequence of “PEST”—SHGFPPEVEEQDDGTLPMSCAQESGMDRHPAACASARINV (SEQ ID NO: 7) motif corresponding to amino acids 422-461 of mouse ornithine decarboxylase was obtained from GenBank (accession number NM_013614.2). Codon optimization and gene synthesis was done by GenScript (Piscataway, NJ), and subcloned into the 3′ end of VH by PCR. The constructs were subcloned into EcoRI and XhoI sites of the MSCV-IRES-GFP vector.

Cloning of scFv Against Human CD7

The sequence scFv derived from murine TH69 (anti-CD7) antibody was obtained from literature (Peipp et al., Cancer Res 2002 (62): 2848-2855). After codon optimization, the synthesized gene consisting of CD8 signal peptide, scFv against human CD7, Myc tag and KDEL (SEQ ID NO: 4) sequence was subcloned into EcoRI and XhoI sites of the MSCV-IRES-GFP vector. Constructs in which myc-KDEL was replaced by other sequences were also made as listed in FIG. 2.

Cloning of scFv Against Human Beta-2 Microglobulin (hB2MG)

The sequence scFv derived from murine BBM.1 (anti-hB2MG) IgG2b antibody was obtained from literature (Grovender, E. A. et al., Kidney Int. 2004;65(1):310-322). After codon optimization, synthesized gene consists of CD8 signal peptide, scFv against human B2MG, Myc tag and KDEL (SEQ ID NO: 4) sequence was subcloned into EcoRI and XhoI sites of the MSCV-IRES-GFP vector.

Cloning of scFv Against Human KIR2DL1 and KIR2DL2/DL3

The amino acid sequence of human monoclonal antibody I-7F9 (anti-KIR2DL1, KIR2DL2, and KIR2DL3) was derived from published International Patent Application WO2006003179 A2 by Moretta et al. After codon optimization, the sequence of scFv was designed by connecting variable light (VL) region and variable heavy (VH) region with linker sequence. The synthesized gene consisting of CD8 signal peptide, scFv against human KIRs (KIR2DL1, KIR2DL2 and KIR2DL3), CD8 hinge and transmembrane domain, and KKMP sequence was subcloned into EcoRI and XhoI sites of the MSCV-IRES-GFP vector. Constructs in which KKMP was replaced by other sequences were also made as listed in FIG. 2.

Cloning of scFv Against Human NKG2A

The sequence of murine antibody Z199 (anti-NKG2A) was derived from the published patent by Spee et al. (EP2247619 A1). After codon optimization, the sequence of scFv was designed by connecting variable light (VL) region and variable heavy (VH) region with linker sequence. The synthesized gene consisting of CD8 signal peptide, scFv against human NKG2A, CD8 hinge and transmembrane, and KKMP sequence was subcloned into EcoRI and XhoI sites of the MSCV-IRES-GFP vector. Constructs in which KKMP was replaced by other sequences were also made as listed in FIG. 2. The sequence information for the scFvs generated herein is shown in Table 1. Sequence information for the various components depicted in FIG. 2 is shown in Table 2.

### Anti-CD19-4-1BB-CD3ζ CAR

This CAR was generated as previously described (Imai, C. et al., Leukemia. 2004;18:676-684; Imai, C. et al., Blood. 2005;106:376-383).

TABLE 1
scFv sequence information

| Target | VH amino acid | VL amino acid | VH cDNA | VL cDNA |
|---|---|---|---|---|
| CD3 | EVQLQQSGAELAR | QIVLTQSPAIMSA | GAGGTCCAGCTGCAGCAG | CAAATTGTTCTCACCCAG |
| | PGASVKMSCKAS | SPGEKVTMTCSAS | TCTGGGGCTGAACTGGCA | TCTCCAGCAATCATGTCT |
| | GYTFTRYTMHWV | SSVSYMNWYQQ | AGACCTGGGGCCTCAGTG | GCATCTCCAGGGGAGAA |
| | KQRPGQGLEWIGY | KSGTSPKRWIYDT | AAGATGTCCTGCAAGGCTT | GGTCACCATGACCTGCA |
| | INPSRGYTNYNQK | SKLASGVPAHFR | CTGGCTACACCTTTACTAG | GTGCCAGCTCAAGTGTA |
| | FKDKATLTTDKSS | GSGSGTSYSLTIS | GTACACGATGCACTGGGT | AGTTACATGAACTGGTAC |
| | STAYMQLSSLTSE | GMEAEDAATYYC | AAAACAGAGGCCTGGACA | CAGCAGAAGTCAGGCAC |
| | DSAVYYCARYYD | QQWSSNPFTFGSG | GGGTCTGGAATGGATTGG | CTCCCCCAAAAGATGGA |
| | DHYCLDYWGQGT | TKLEINR (SEQ ID | ATACATTAATCCTAGCCGT | TTTATGACACATCCAAAC |
| | TLTVSSA (SEQ ID | NO: 13) | GGTTATACTAATTACAATC | TGGCTTCTGGAGTCCCTG |
| | NO: 12) | | AGAAGTTCAAGGACAAGG | CTCACTTCAGGGGCAGTG |
| | | | CCACATTGACTACAGACA | GGTCTGGGACCTCTTACT |
| | | | AATCCTCCAGCACAGCCTA | CTCTCACAATCAGCGGCA |
| | | | CATGCAACTGAGCAGCCT | TGGAGGCTGAAGATGCT |
| | | | GACATCTGAGGACTCTGCA | GCCACTTATTACTGCCAG |
| | | | GTCTATTACTGTGCAAGAT | CAGTGGAGTAGTAACCC |
| | | | ATTATGATGATCATTACTG | ATTCACGTTCGGCTCGGG |
| | | | CCTTGACTACTGGGGCCAA | GACAAAGTTGGAAATAA |
| | | | GGCACCACTCTCACAGTCT | ACCGG (SEQ ID NO: |
| | | | CCTCAGCC (SEQ ID NO: | 15) |
| | | | 14) | |
| CD7 | EVQLVESGGGLVK | AAYKDIQMTQTT | GAGGTGCAGCTGGTCGAA | GCCGCATACAAGGATAT |
| (TH69) | PGGSLKLSCAASG | SSLSASLGDRVTIS | TCTGGAGGAGGACTGGTG | TCAGATGACTCAGACCA |
| | LTFSSYAMSWVR | CSASQGISNYLN | AAGCCAGGAGGATCTCTG | CAAGCTCCCTGAGCGCCT |
| | QTPEKRLEWVASI | WYQQKPDGTVKL | AAACTGAGTTGTGCCGCTT | CCCTGGGAGACCGAGTG |
| | SSGGFTYYPDSVK | LIYYTSSLHSGVP | CAGGCCTGACCTTCTCAAG | ACAATCTCTTGCAGTGCA |
| | GRFTISRDNARNIL | SRFSGSGSGTDYS | CTACGCCATGAGCTGGGTG | TCACAGGGAATTAGCAA |
| | YLQMSSLRSEDTA | LTISNLEPEDIATY | CGACAGACACCTGAGAAG | CTACCTGAATTGGTATCA |
| | MYYCARDEVRGY | YCQQYSKLPYTF | CGGCTGGAATGGGTCGCT | GCAGAAGCCAGATGGCA |
| | LDVWGAGTTVTV | GGGTKLEIKR | AGCATCTCCTCTGGCGGGT | CTGTGAAACTGCTGATCT |
| | SS (SEQ ID NO: 16) | (SEQ ID NO: 17) | TCACATACTATCCAGACTC | ACTATACCTCTAGTCTGC |
| | | | CGTGAAAGGCAGATTTACT | ACAGTGGGGTCCCCTCAC |
| | | | ATCTCTCGGGATAACGCAA | GATTCAGCGGATCCGGCT |
| | | | GAAATATTCTGTACCTGCA | CTGGGACAGACTACAGC |
| | | | GATGAGTTCACTGAGGAG | CTGACTATCTCCAACCTG |
| | | | CGAGGACACCGCAATGTA | GAGCCCGAAGATATTGC |
| | | | CTATTGTGCCAGGGACGA | CACCTACTATTGCCAGCA |
| | | | AGTGCGCGGCTATCTGGAT | GTACTCCAAGCTGCCTTA |
| | | | GTCTGGGGAGCTGGCACT | TACCTTTGGCGGGGGAA |
| | | | ACCGTCACCGTCTCCAGC | CAAAGCTGGAGATTAAA |
| | | | (SEQ ID NO: 18) | AGG (SEQ ID NO: 19) |
| CD7 | QVQLQESGAELVK | DIELTQSPATLSVT | CAGGTCCAGCTGCAGGAG | GACATCGAGCTGACACA |
| (3a1f) | PGASVKLSCKASG | PGDSVSLSCRASQ | TCAGGGGCAGAGCTGGTG | GTCTCCAGCCACTCTGAG |
| | YTFTSYWMHWVK | SISNNLHWYQQK | AAACCCGGAGCCAGTGTC | CGTGACCCCTGGCGATTC |
| | QRPGQGLEWIGKI | SHESPRLLIKSASQ | AAACTGTCCTGTAAGGCCA | TGTCAGTCTGTCATGTAG |
| | NPSNGRTNYNEKF | SISGIPSRFSGSGS | GCGGCTATACTTTCACCAG | AGCTAGCCAGTCCATCTC |
| | KSKATLTVDKSSS | GTDFTLSINSVETE | CTACTGGATGCACTGGGTG | TAACAATCTGCACTGGTA |
| | TAYMQLSSLTSED | DFGMYFCQQSNS | AAACAGAGGCCAGGACAG | CCAGCAGAAATCACATG |
| | SAVYYCARGGVY | WPYTFGGGTKLEI | GGCCTGGAGTGGATCGGC | AAAGCCCTCGGCTGCTG |
| | YDLYYYALDYWG | KR (SEQ ID NO: | AAGATTAACCCCAGCAAT | ATTAAGAGTGCTTCACAG |
| | QGTTVTVSS (SEQ | 21) | GGGCGCACCAACTACAAC | AGCATCTCCGGGATTCCA |
| | ID NO: 20) | | GAAAAGTTTAAATCCAAG | AGCAGATTCTCTGGCAGT |
| | | | GCTACACTGACTGTGGACA | GGGTCAGGAACCGACTT |
| | | | AGAGCTCCTCTACCGCATA | TACACTGTCCATTAACTC |
| | | | CATGCAGCTGAGTTCACTG | TGTGGAGACCGAAGATT |
| | | | ACATCTGAAGATAGTGCC | TCGGCATGTATTTTTGCC |
| | | | GTGTACTATTGCGCCAGAG | AGCAGAGCAATTCCTGG |
| | | | GCGGGGTCTACTATGACCT | CCTTACACATTCGGAGGC |
| | | | GTACTATTACGCACTGGAT | GGGACTAAACTGGAGAT |
| | | | TATTGGGGGCAGGGAACC | TAAGAGG (SEQ ID NO: |
| | | | ACAGTGACTGTCAGCTCC | 23) |
| | | | (SEQ ID NO: 22) | |
| CD45 | QVQLVESGGGLV | DIVLTQSPASLAV | CAGGTGCAGCTGGTCGAG | GACATTGTGCTGACCCAG |
| | QPGGSLKLSCAAS | SLGQRATISCRAS | TCTGGAGGAGGACTGGTG | TCCCCTGCTTCACTGGCA |
| | GFDFSRYWMSWV | KSVSTSGYSYLH | CAGCCTGGAGGAAGTCTG | GTGAGCCTGGGACAGAG |
| | RQAPGKGLEWIGE | WYQQKPGQPPKL | AAGCTGTCATGTGCAGCCA | GGCAACCATCAGCTGCC |
| | INPTSSTINFTPSLK | LIYLASNLESGVP | GCGGGTTCGACTTTTCTCG | GAGCCTCTAAGAGTGTCT |
| | DKVFISRDNAKNT | ARFSGSGSGTDFT | ATACTGGATGAGTTGGGTG | CAACAAGCGGATACTCC |
| | LYLQMSKVRSEDT | LNIHPVEEEDAAT | CGGCAGGCACCAGGAAAA | TATCTGCACTGGTACCAG |
| | ALYYCARGNYYR | YYCQHSRELPFTF | GGACTGGAATGGATCGGC | CAGAAGCCAGGACAGCC |
| | YGDAMDYWGQG | GSGTKLEIK (SEQ | GAGATTAACCCAACTAGCT | ACCTAAACTGCTGATCTA |
| | TSVTVS (SEQ ID | ID NO: 25) | CCACCATCAATTTCACACC | TCTGGCTTCCAACCTGGA |
| | NO: 24) | | CAGCCTGAAGGACAAAGT | ATCTGGAGTGCCTGCACG |
| | | | GTTTATTTCCAGAGATAAC | CTTCTCCGGATCTGGAAG |
| | | | GCCAAGAATACTCTGTATC | TGGAACCGACTTTACACT |
| | | | TGCAGATGTCCAAAGTCA | GAATATTCACCCAGTCGA |
| | | | GGTCTGAAGATACCGCCCT | GGAAGAGGATGCCGCTA |
| | | | GTACTATTGTGCTCGGGGC | CCTACTATTGCCAGCACA |
| | | | AACTACTATAGATACGGG | GCCGGGAGCTGCCCTTCA |
| | | | GACGCTATGGATTATTGGG | CATTTGGCAGCGGGACT |
| | | | GGCAGGGAACTAGCGTGA | AAGCTGGAGATCAAG |
| | | | CCGTGAGT (SEQ ID NO: | (SEQ ID NO: 27) |
| | | | 26) | |
| B2MG | EVQLQQSGAELVK | DIQMTQSPASQSA | GAGGTGCAGCTGCAGCAG | GATATTCAGATGACCCA |
| | PGASVKLSCTPSG | SLGESVTITCLAS | AGCGGAGCAGAACTGGTG | GTCCCCTGCATCACAGAG |
| | FNVKDTYIHWVK | QTIGTWLAWYQQ | AAACCTGGAGCCAGCGTC | CGCCTCCCTGGGCGAGTC |
| | QRPKQGLEWIGRI | KPGKSPQLLIYAA | AAGCTGTCCTGTACTCCAT | AGTGACCATCACATGCCT |
| | DPSDGDIKYDPKF | TSLADGVPSRFSG | CTGGCTTCAACGTGAAGG | GGCTAGCCAGACAATTG |
| | QGKATITADTSSN | SGSGTKFSLKIRT | ACACATACATTCACTGGGT | GCACTTGGCTGGCATGGT |
| | TVSLQLSSLTSEDT | LQAEDFVSYYCQ | CAAGCAGCGGCCCAAACA | ACCAGCAGAAGCCCGGC |
| | AVYYCARWFGDY | QLYSKPYTFGGG | GGGACTGGAGTGGATCGG | AAATCCCCTCAGCTGCTG |
| | GAMNYWGQGTSV | TKLEIKRAD (SEQ | CAGAATTGACCCATCCGAC | ATCTATGCAGCTACCTCT |
| | TVSS (SEQ ID NO: | ID NO: 29) | GGCGATATCAAGTATGATC | CTGGCAGACGGAGTGCC |
| | 28) | | CCAAATTCCAGGGGAAGG | CAGTAGGTTCTCTGGGAG |
| | | | CTACTATTACCGCAGATAC | TGGATCAGGCACCAAGT |
| | | | CAGCTCCAACACAGTGAG | TTTCTCTGAAAATTCGCA |
| | | | TCTGCAGCTGTCTAGTCTG | CACTGCAGGCTGAGGAT |
| | | | ACTAGCGAAGACACCGCC | TTCGTCTCCTACTATTGC |
| | | | GTCTACTATTGTGCTAGAT | CAGCAGCTGTACTCTAAA |
| | | | GGTTTGGCGATTACGGGGC | CCTTATACATTTGGCGGG |
| | | | CATGAATTATTGGGGGCA | GGAACTAAGCTGGAAAT |
| | | | GGGAACCAGCGTCACCGT | CAAACGAGCAGAC (SEQ |
| | | | GTCCAGC (SEQ ID NO: | ID NO: 31) |
| | | | 30) | |
| NKG2A | EVQLVESGGGLVK | QIVLTQSPALMSA | GAGGTGCAGCTGGTGGAG | CAGATTGTCCTGACCCAG |
| | PGGSLKLSCAASG | SPGEKVTMTCSAS | AGCGGAGGAGGACTGGTG | TCTCCAGCCCTGATGAGC |
| | FTFSSYAMSWVRQ | SSVSYIYWYQQK | AAGCCAGGAGGAAGCCTG | GCCTCCCCTGGCGAGAA |
| | SPEKRLEWVAEISS | PRSSPKPWIYLTS | AAGCTGTCCTGTGCCGCCT | GGTGACAATGACCTGCTC |
| | GGSYTYYPDTVTG | NLASGVPARFSGS | CTGGCTTCACATTTTCCTC | TGCCAGCTCCTCTGTGAG |
| | RFTISRDNAKNTL | GSGTSYSLTISSM | TTATGCAATGAGCTGGGTG | CTACATCTATTGGTACCA |
| | YLEISSLRSEDTAM | EAEDAATYYCQQ | CGGCAGTCCCCAGAGAAG | GCAGAAGCCTCGGAGCT |
| | YYCTRHGDYPRFF | WSGNPYTFGGGT | AGACTGGAGTGGGTGGCA | CCCCAAAGCCCTGGATCT |
| | DVWGAGTTVTVS | KLEIKR (SEQ ID | GAGATCAGCTCCGGAGGA | ATCTGACATCCAACCTGG |
| | S (SEQ ID NO: 32) | NO: 33) | TCCTACACCTACTATCCTG | CCTCTGGCGTGCCAGCCA |
| | | | ACACAGTGACCGGCCGGT | GATTCTCTGGCAGCGGCT |
| | | | TCACAATCTCTAGAGATAA | CCGGCACATCTTACAGCC |
| | | | CGCCAAGAATACCCTGTAT | TGACCATCTCTAGCATGG |
| | | | CTGGAGATCTCTAGCCTGA | AGGCCGAGGACGCCGCC |
| | | | GATCCGAGGATACAGCCA | ACCTACTATTGCCAGCAG |
| | | | TGTACTATTGCACCAGGCA | TGGTCCGGCAATCCATAT |
| | | | CGGCGACTACCCACGCTTC | ACATTTGGCGGCGGCAC |
| | | | TTTGACGTGTGGGGAGCA | CAAGCTGGAGATCAAGA |
| | | | GGAACCACAGTGACCGTG | GG (SEQ ID NO: 35) |
| | | | TCCTCT (SEQ ID NO: | |
| | | | 34) | |
| KIR | QVQLVQSGAEVK | EIVLTQSPVTLSLS | CAGGTCCAGCTGGTGCAGT | GAGATCGTGCTGACCCA |
| 2DL1 | KPGSSVKVSCKAS | PGERATLSCRASQ | CTGGAGCTGAAGTGAAGA | GTCTCCTGTCACACTGAG |
| and 2/3 | GGTFSFYAISWVR | SVSSYLAWYQQK | AACCAGGGAGCTCCGTCA | TCTGTCACCAGGGGAAC |
| | QAPGQGLEWMGG | PGQAPRLLIYDAS | AGGTGTCATGCAAAGCAA | GGGCTACACTGTCTTGCA |
| | FIPIFGAANYAQKF | NRATGIPARFSGS | GCGGCGGGACTTTCTCCTT | GAGCAAGCCAGTCCGTG |
| | QGRVTITADESTST | GSGTDFTLTISSLE | TTATGCAATCTCTTGGGTG | AGCTCCTACCTGGCCTGG |
| | AYMELSSLRSDDT | PEDFAVYYCQQR | AGACAGGCACCTGGACAG | TATCAGCAGAAGCCAGG |
| | AVYYCARiPSGSY | SNWMYTFGQGTK | GGACTGGAGTGGATGGGA | CCAGGCTCCCAGGCTGCT |
| | YYDYDMDVWGQ | LEIKRT (SEQ ID | GGCTTCATCCCAATTTTTG | GATCTACGATGCAAGCA |
| | GTTVTVSS (SEQ | NO: 37) | GAGCCGCTAACTATGCCCA | ACAGGGCCACTGGGATT |
| | ID NO: 36) | | GAAGTTCCAGGGCAGGGT | CCCGCCCGCTTCTCTGGC |
| | | | GACCATCACAGCTGATGA | AGTGGGTCAGGAACCGA |
| | | | GTCTACTAGTACCGCATAC | CTTTACTCTGACCATTTC |
| | | | ATGGAACTGTCTAGTCTGA | TAGTCTGGAGCCTGAAG |
| | | | GGAGCGACGATACCGCCG | ATTTCGCCGTGTACTATT |
| | | | TGTACTATTGTGCTCGCAT | GCCAGCAGCGATCCAAT |
| | | | TCCATCAGGCAGCTACTAT | TGGATGTATACTTTTGGC |
| | | | TACGACTATGATATGGACG | CAGGGGACCAAGCTGGA |
| | | | TGTGGGGCCAGGGGACCA | GATCAAACGGACA (SEQ |
| | | | CAGTCACCGTGAGCAGC | ID NO: 39) |
| | | | (SEQ ID NO: 38) | |

TABLE 2
Sequence information for components depicted in FIG. 2

| | CD8 SP | VH-VL | | | | |
|---|---|---|---|---|---|---|
| | amino | linker | CD8 hinge and | | VH-VL linker | CD8 hinge and TM |
| Target | acid | amino acid | TM amino acid | CD8 SP cDNA | cDNA | cDNA |
| CD3 | MALPVT | GGGGSGG | KPTTTPAPRPPTP | ATGGCCTTACC | GGTGGTGGTG | AAGCCCACCACG |
| | ALLLPLA | GGSGGGG | APTIASQPLSLRP | AGTGACCGCCT | GTTCTGGTGG | ACGCCAGCGCCG |
| | LLLHAAR | SGGGGS | EACRPAAGGAVH | TGCTCCTGCCG | TGGTGGTTCT | CGACCACCAACA |
| | P (SEQ ID | (SEQ ID | TRGLDFACDIYI | CTGGCCTTGCT | GGCGGCGGCG | CCGGCGCCCACC |
| | NO: 40) | NO: 41) | WAPLAGTCGVLL | GCTCCACGCCG | GCTCCGGTGG | ATCGCGTCGCAG |
| | | | LSLVITLY (SEQ | CCAGGCCG | TGGTGGATCC | CCCCTGTCCCTGC |
| | | | ID NO: 42) | (SEQ ID NO: | (SEQ ID NO: | GCCCAGAGGCGT |
| | | | | 44) | 51) | GCCGGCCAGCGG |
| | | | | | | CGGGGGGCGCAG |
| | | | | | | TGCACACGAGGG |
| | | | | | | GGCTGGACTTCG |
| | | | | | | CCTGTGATATCTA |
| | | | | | | CATCTGGGCGCC |
| | | | | | | CTTGGCCGGGAC |
| | | | | | | TTGTGGGGTCCTT |
| | | | | | | CTCCTGTCACTGG |
| | | | | | | TTATCACCCTTTA |
| | | | | | | C (SEQ ID NO: |
| | | | | | | 57) |
| CD7 | MALPVT | GGGGSGG | TTTPAPRPPTPAP | ATGGCTCTGCC | GGAGGAGGAG | ACCACTACACCT |
| (TH69) | ALLLPLA | GGSGGGG | TIASQPLSLRPEA | TGTGACCGCAC | GAAGCGGAGG | GCACCAAGGCCT |
| | LLLHAAR | SGGGGS | CRPAAGGAVHTR | TGCTGCTGCCC | AGGAGGATCC | CCCACACCCGCTC |
| | P (SEQ ID | (SEQ ID | GLDFACDIYIWA | CTGGCTCTGCT | GGAGGCGGGG | CCACTATCGCTTC |
| | NO: 40) | NO: 41) | PLAGTCGVLLLS | GCTGCACGCCG | GATCTGGAGG | CCAGCCACTGTCC |
| | | | LVITLY (SEQ ID | CAAGACCT | AGGAGGAAGT | CTGAGGCCCGAG |
| | | | NO: 50) | (SEQ ID NO: | (SEQ ID NO: | GCCTGCAGGCCA |
| | | | | 45) | 52) | GCAGCTGGCGGA |
| | | | | | | GCCGTGCATACT |
| | | | | | | AGGGGGCTGGAC |
| | | | | | | TTCGCTTGCGACA |
| | | | | | | TCTACATCTGGGC |
| | | | | | | CCCACTGGCAGG |
| | | | | | | GACATGCGGAGT |
| | | | | | | CCTGCTGCTGTCC |
| | | | | | | CTGGTCATCACAC |
| | | | | | | TTTAC (SEQ ID |
| | | | | | | NO: 58) |
| CD7 | MALPVT | GGGGSGG | TTTPAPRPPTPAP | ATGGCTCTGCC | GGAGGAGGAG | ACTACCACACCA |
| (3a1f) | ALLLPLA | GGSGGGG | TIASQPLSLRPEA | CGTCACCGCTC | GATCCGGCGG | GCTCCAAGACCA |
| | LLLHAAR | S (SEQ ID | CRPAAGGAVHTR | TGCTGCTGCCT | AGGAGGCTCT | CCTACCCCTGCAC |
| | P (SEQ ID | NO: 43) | GLDFACDIYIWA | CTGGCTCTGCT | GGGGGAGGCG | CAACAATTGCTA |
| | NO: 40) | | PLAGTCGVLLLS | GCTGCACGCTG | GGAGT (SEQ | GTCAGCCACTGTC |
| | | | LVITLY (SEQ ID | CTCGACCA | ID NO: 53) | ACTGAGACCAGA |
| | | | NO: 50) | (SEQ ID NO: | | AGCATGTAGGCC |
| | | | | 46) | | TGCAGCTGGAGG |
| | | | | | | AGCTGTGCACAC |
| | | | | | | CAGAGGCCTGGA |
| | | | | | | CTTTGCCTGCGAT |
| | | | | | | ATCTACATTTGGG |
| | | | | | | CTCCTCTGGCAGG |
| | | | | | | AACCTGTGGCGT |
| | | | | | | GCTGCTGCTGTCT |
| | | | | | | CTGGTCATCACAC |
| | | | | | | TTTAC (SEQ ID |
| | | | | | | NO: 59) |
| CD45 | MALPVT | GGGGSGG | KPTTTPAPRPPTP | ATGGCTCTGCC | GGAGGAGGAG | AAGCCCACCACG |
| | ALLLPLA | GGSGGGG | APTIASQPLSLRP | CGTGACCGCTC | GAAGTGGAGG | ACGCCAGCGCCG |
| | LLLHAAR | SGGGGS | EACRPAAGGAVH | TGCTGCTGCCT | AGGAGGATCA | CGACCACCAACA |
| | P (SEQ ID | (SEQ ID | TRGLDFACDIYI | CTGGCTCTGCT | GGAGGCGGGG | CCGGCGCCCACC |
| | NO: 40) | NO: 41) | WAPLAGTCGVLL | GCTGCATGCTG | GAAGCGGCGG | ATCGCGTCGCAG |
| | | | LSLVITLY (SEQ | CTCGACCT | GGGAGGCTCC | CCCCTGTCCCTGC |
| | | | ID NO: 42) | (SEQ ID NO: | (SEQ ID NO: | GCCCAGAGGCGT |
| | | | | 47) | 54) | GCCGGCCAGCGG |
| | | | | | | CGGGGGGCGCAG |
| | | | | | | TGCACACGAGGG |
| | | | | | | GGCTGGACTTCG |
| | | | | | | CCTGTGATATCTA |
| | | | | | | CATCTGGGCGCC |
| | | | | | | CTTGGCCGGGAC |
| | | | | | | TTGTGGGGTCCTT |
| | | | | | | CTCCTGTCACTGG |
| | | | | | | TTATCACCCTTTA |
| | | | | | | C (SEQ ID NO: |
| | | | | | | 57) |
| B2MG | MALPVT | GGGGSGG | KPTTTPAPRPPTP | ATGGCCCTGCC | GGAGGAGGAG | AAGCCCACCACG |
| | ALLLPLA | GGSGGGG | APTIASQPLSLRP | CGTCACCGCCC | GAAGTGGAGG | ACGCCAGCGCCG |
| | LLLHAAR | SGGGGS | EACRPAAGGAVH | TGCTGCTGCCC | AGGAGGGTCA | CGACCACCAACA |
| | P (SEQ ID | (SEQ ID | TRGLDFACDIYI | CTGGCTCTGCT | GGAGGCGGGG | CCGGCGCCCACC |
| | NO: 40) | NO: 41) | WAPLAGTCGVLL | GCTGCACGCCG | GAAGCGGCGG | ATCGCGTCGCAG |
| | | | LSLVITLY (SEQ | CAAGACCC | GGGAGGATCC | CCCCTGTCCCTGC |
| | | | ID NO: 42) | (SEQ ID NO: | (SEQ ID NO: | GCCCAGAGGCGT |
| | | | | 48) | 55) | GCCGGCCAGCGG |
| | | | | | | CGGGGGGCGCAG |
| | | | | | | TGCACACGAGGG |
| | | | | | | GGCTGGACTTCG |
| | | | | | | CCTGTGATATCTA |
| | | | | | | CATCTGGGCGCC |
| | | | | | | CTTGGCCGGGAC |
| | | | | | | TTGTGGGGTCCTT |
| | | | | | | CTCCTGTCACTGG |
| | | | | | | TTATCACCCTTTA |
| | | | | | | C (SEQ ID NO: |
| | | | | | | 57) |
| NKG2A | MALPVT | GGGGSGG | KPTTTPAPRPPTP | ATGGCTCTGCC | GGAGGAGGAG | AAGCCAACCACA |
| | LLLPLAA | GGSGGGG | APTIASQPLSLRP | CGTGACCGCCC | GATCTGGAGG | ACCCCTGCACCA |
| | LLLHAAR | SGGGGS | EACRPAAGGAVH | TGCTGCTGCCT | AGGAGGCAGC | AGGCCACCTACA |
| | P (SEQ ID | (SEQ ID | TRGLDFACDIYI | CTGGCTCTGCT | GGCGGCGGCG | CCAGCACCTACC |
| | NO: 40) | NO: 41) | WAPLAGTCGVLL | GCTGCACGCTG | GCTCCGGCGG | ATCGCAAGCCAG |
| | | | LSLVITLY (SEQ | CCCGCCCA | CGGCGGCTCT | CCACTGTCCCTGA |
| | | | ID NO: 42) | (SEQ ID NO: | (SEQ ID NO: | GGCCAGAGGCAT |
| | | | | 49) | 56) | GTAGGCCTGCAG |
| | | | | | | CAGGAGGCGCCG |
| | | | | | | TGCACACACGCG |
| | | | | | | GCCTGGACTTTGC |
| | | | | | | CTGCGATATCTAC |
| | | | | | | ATCTGGGCACCA |
| | | | | | | CTGGCAGGAACC |
| | | | | | | TGTGGCGTGCTGC |
| | | | | | | TGCTGAGCCTGGT |
| | | | | | | GATTACCCTGTAT |
| | | | | | | (SEQ ID NO: |
| | | | | | | 60) |
| KIR | MALPVT | GGGGSGG | KPTTTPAPRPPTP | ATGGCCTTACC | GGTGGTGGTG | AAGCCCACCACG |
| 2DL1 | ALLLPLA | GGSGGGG | APTIASQPLSLRP | AGTGACCGCCT | GTTCTGGTGG | ACGCCAGCGCCG |
| and 2/3 | LLLHAAR | SGGGGS | EACRPAAGGAVH | TGCTCCTGCCG | TGGTGGTTCT | CGACCACCAACA |
| | P (SEQ ID | (SEQ ID | TRGLDFACDIYI | CIGGCcrTGur | GGCGGCGGCG | CCGGCGCCCACC |
| | NO: 40) | NO: 41) | WAPLAGTCGVLL | GCTCCACGCCG | GCTCCGGTGG | ATCGCGTCGCAG |
| | | | LSLVITLY (SEQ | CCAGGCCG | TGGTGGATCC | CCCCTGTCCCTGC |
| | | | ID NO: 42) | (SEQ ID NO: | (SEQ ID NO: | GCCCAGAGGCGT |
| | | | | 44) | 51) | GCCGGCCAGCGG |
| | | | | | | CGGGGGGCGCAG |
| | | | | | | TGCACACGAGGG |
| | | | | | | GGCTGGACTTCG |
| | | | | | | CCTGTGATATCTA |
| | | | | | | CATCTGGGCGCC |
| | | | | | | CTTGGCCGGGAC |
| | | | | | | TTGTGGGGTCCTT |
| | | | | | | CTCCTGTCACTGG |
| | | | | | | TTATCACCCTTTA |
| | | | | | | C (SEQ ID NO: |
| | | | | | | 57) |

Gene transduction, cell expansion, flow cytometric analysis and functional studies

These were performed as previously described (Kudo, K et al., Cancer Res. 2014;74(1):93-103).

### Results

Generation of scFv Constructs

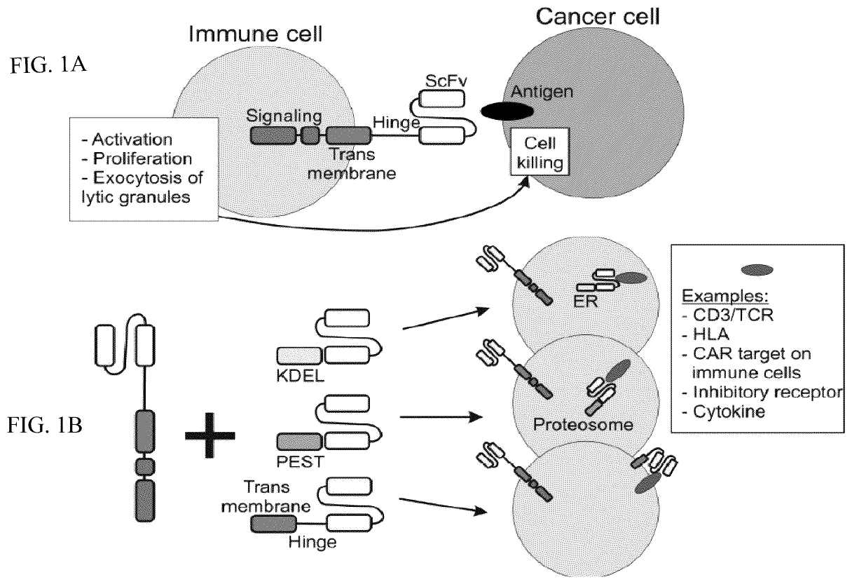

A schematic of the technology is outlined in FIG. 1. A schematic representation of the inhibitory constructs that we generated is shown in FIG. 2. The scFv portion can be derived by cloning the cDNA encoding variable light (VL) and variable heavy (VH) immunoglobulin chain regions from an antibody-producing hybridoma cell line or from the corresponding published sequences. VL and VH are linked with a short peptide sequence (“linker”) according to standard techniques to make a full scFv. To be expressed, the scFv is linked to a signal peptide at the N-terminus; the signal peptide is required for the scFv to be expressed, as confirmed in preliminary experiments. Proteins containing scFv plus signal peptide are generally released into the cells' milieu. For example, in preliminary experiments (not shown), an anti-CD3ε scFv plus signal peptide expressed in Jurkat T cells was detected in the cells' culture supernatant. By directing the scFv to specific compartments and preventing its secretion, possible effects on other cells are prevented. To direct it to the endoplasmic reticulum (ER), the KDEL (SEQ ID NO: 4) motif (which retains proteins in the ER) was utilized (Strebe N. et al., J Immunol Methods. 2009;341(1-2):30-40). To promote the degradation of the targeted protein, we linked it to a proteasome-targeting PEST motif (Joshi, S. N. et al.,MAbs. 2012;4(6):686-693). The scFv can also be directed to the cell membrane by linking it to the transmembrane domain and hinge of CD8α or another transmembrane protein.

Downregulation of T-cell Receptor in T Lymphocytes Expressing anti-CD19-BB-ζ CAR

To determine whether the proposed strategy could be applied to generate immune cells expressing CAR and lacking one or more markers, T-cell receptor (TCR) expression was downregulated in anti-CD19 CAR T-cells.

To be expressed on the cell membrane, the CD3/TCR complex requires assembly of all its components (TCRα, TCRβ, CD3δ, CD3ε, CD3γ, CD3ζ). Lack of one component prevents CD3/TCR expression and, therefore, antigen recognition. In preliminary studies, the scFv from an anti-CD3ε hybridoma (purchased from Creative Diagnostics, Shirley, NY) was cloned and generated the constructs containing KDEL (SEQ ID NO: 4), PEST, CD8α transmembrane domain or others as shown in FIG. 2.

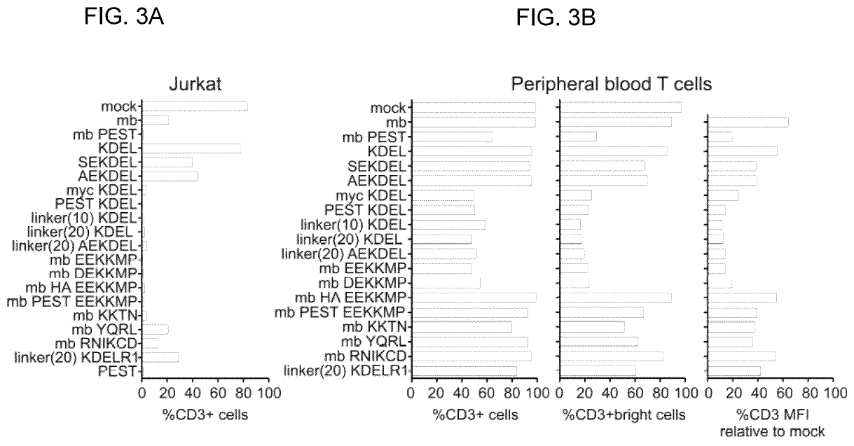

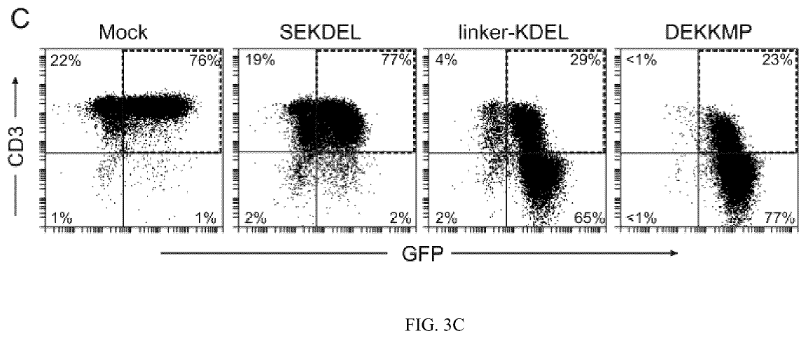

The constructs disclosed herein were transduced in the CD3/TCR+ Jurkat cell line using a murine stem cell virus (MSCV) retroviral vector containing green fluorescent protein (GFP). Percentage of GFP+ cells after transduction was >90% in all experiments. FIG. 3A shows results of staining with anti-CD3ε antibody among GFP+ cells, as measured by flow cytometry. Antibody staining of CD3ε was decreased to variable degree in cells transduced with the constructs listed. Similar downregulation of CD3ε was obtained with human peripheral blood T lymphocytes (FIG. 3B). FIG. 3C shows illustrative flow cytometry dot plots of CD3ε expression in GFP-positive Jurkat cells after transduction with different gene constructs in comparison with cells transduced with a vector containing GFP alone.

Downregulation of CD3 did not affect growth of Jurkat cells or expression of all other cell markers tested, including CD2, CD4, CD8, CD45, CD25, CD69. Inhibition of CD3 expression persisted for over 3 months. Further enrichment of CD3-negative cells could be achieved by CD3+ T cell depletion with anti-CD3 magnetic beads (Dynal, Life Technologies, Carlsbad, CA).

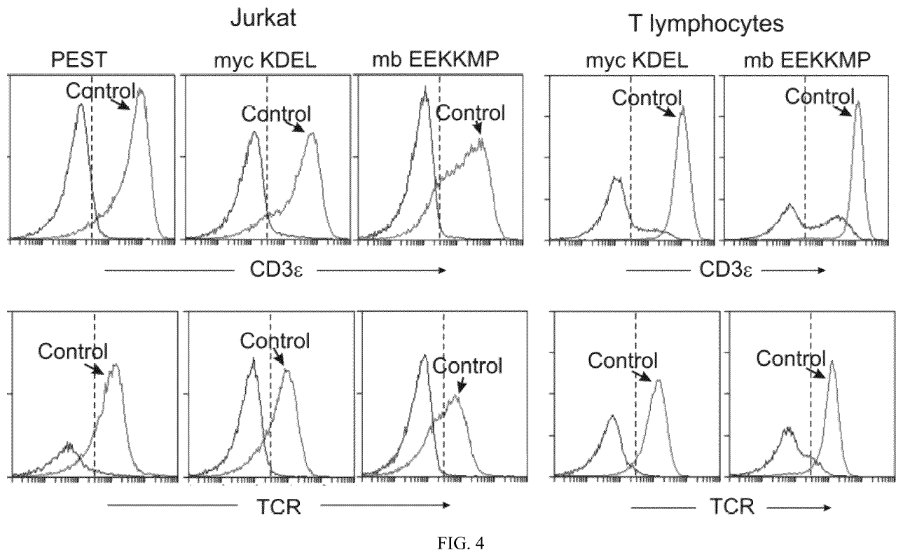

Staining with anti-TCRαβ antibody of Jurkat cells or human peripheral blood T lymphocytes showed that down regulation of CD3ε expression was associated with dowregulation of TCRαβ expression (FIG. 4).

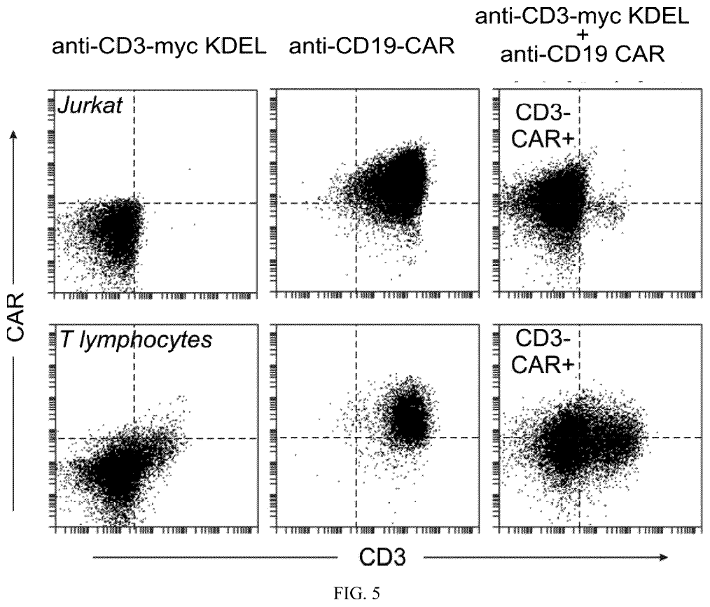

Next, it was determined whether the anti-CD3 scFv-myc KDEL could be expressed simultaneously with an anti-CD19-4-1BB-CD3ζ CAR. As shown in FIG. 5, this resulted in T cells lacking CD3 expression while expressing the anti-CD19 CAR. TCR was also absent on these cells (not shown).

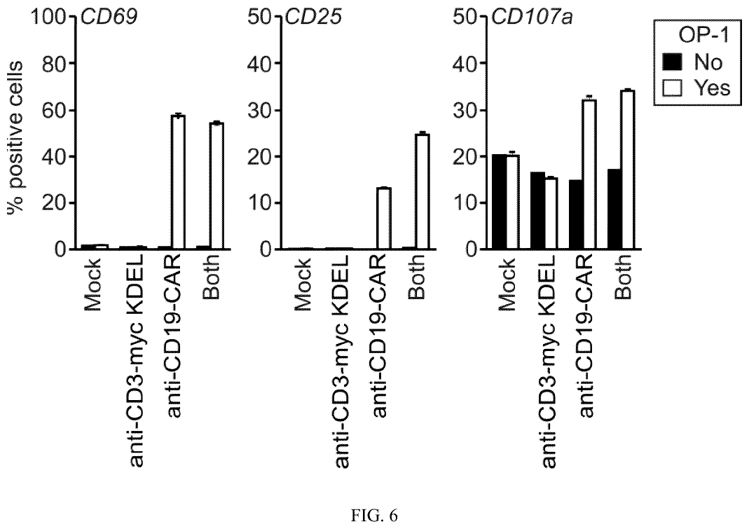

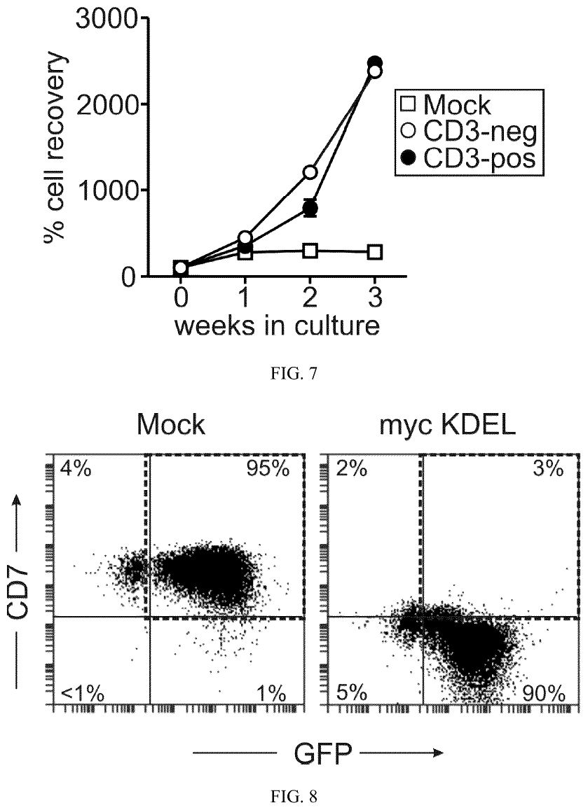

To assess whether CAR could signal in Jurkat cells with downregulated CD3/TCR, the expression of the activation markers CD69 and CD25 was tested, and exocytosis of lytic granules was measured by CD107a expression in Jurkat cells co-cultured with the CD19+ leukemia cell line OP-1. As shown in FIG. 6, downregulation of CD3/TCR with the anti-CD3 scFv-myc KDEL construct did not diminish the capacity of anti-CD19-4-1BB-CD3ζ CAR to activate Jurkat cells. To further explore the effects of CD3/TCR deletion on CAR signaling, it was determined whether CD3-negative T lymphocytes expressing the CAR could be stimulated by its ligation. As shown in FIG. 7, co-culture of T lymphocytes expressing the anti-CD19 CAR with CD19+ leukemic cells led to T cell proliferation regardless of whether CD3 was downregulated or not, indicating that CD3/TCR downregulation did not diminish the CAR proliferative stimulus.

Accordingly, CD3/TCR can be effectively downregulated in CAR-T cells using the anti-CD3 scFv-myc KDEL construct without affecting T cell activation, degranulation and proliferation driven by the CAR.

### Downregulation of CD7

It was determined whether the strategy that successfully modulated CD3/TCR expression could be applied to other surface molecules. For this purpose, CD7 expression was modulated. The scFv sequence was derived from that published by Peipp et al. (Cancer Res 2002 (62): 2848-2855), which was linked to the CD8 signal peptide and the myc-KDEL sequence as illustrated in FIG. 2. Using the MSCV retroviral vector, the anti-CD7-myc KDEL construct was transduced in peripheral blood lymphocytes, which have high expression of CD7 as detected by an anti-CD7 antibody conjugated to phycoerythrin (BD Bioscience). As shown in FIG. 8, CD7 in T lymphocytes transduced with the construct was virtually abrogated.

### Downregulation of HLA-Class I

The strategy was then applied to downregulate another surface molecule, HLA class I.

HLA class I consists of polymorphic a chains and a non-polymorphic chain termed β2-microglobulin. Knock-down of the latter subunit results in abrogation of HLA (MHC in the mouse) Class I expression (Koller, B H et al., Science. 1990;248(4960):1227-1230). An scFv reacting with β2-microglobulin was used to suppress expression of HLA Class I in immune cells.

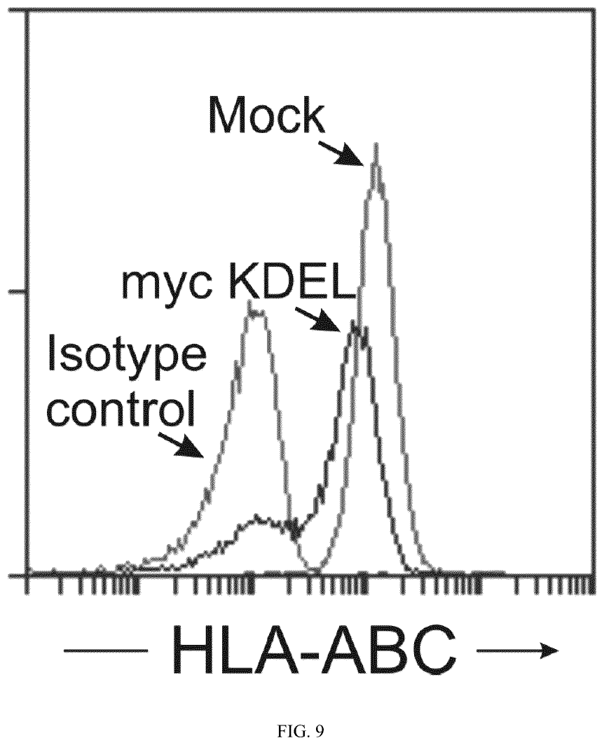

The scFv sequence was derived from that published by Grovender et al. (Kidney Int. 2004;65(1):310-322), which was linked to the CD8 signal peptide and the myc KDEL sequence as illustrated in FIG. 2. Using the MSCV retroviral vector, the anti-β2M-myc KDEL construct was transduced in Jurkat cells, which have high expression of HLA Class I as detected by an anti-HLA-ABC antibody conjugated to phycoerythrin (BD Pharmingen). As shown in FIG. 9, Jurkat cells transduced with the construct had a substantial downregulation of HLA-ABC expression. Cells maintained their morphology and growth capacity.

### Dowregulation of Inhibitory Receptors in NK Cells

To determine if the strategy outlined above would also apply to surface molecules expressed in other immune cells, downregulation of function of the inhibitory receptor KIR2DL1, KIR2DL2/DL3 and NKG2A was tested in NK cells.

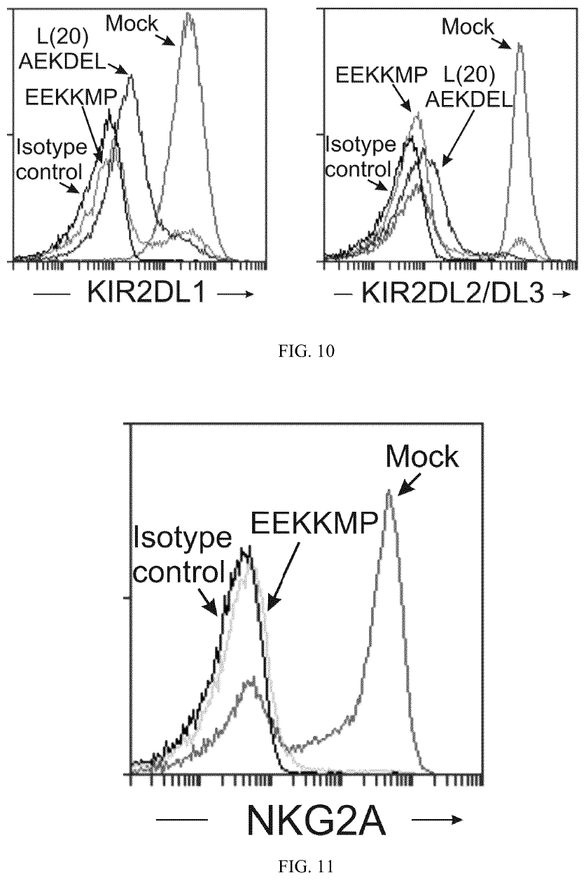

To downregulate KIR receptors, an scFv reacting with KIR2DL1 and KIR2DL2/DL3 was used to suppress their expression in NK cells. The scFv sequence was derived from that published by Moretta et al. (patent WO2006003179 A2), which was linked to the CD8 signal peptide and the ER retention sequences as illustrated in FIG. 2. Using the MSCV retroviral vector, the constructs were transduced in NK cells expanded from human peripheral blood and selected for KIR2DL1 expression. These cells had high KIR2DL1 expression as detected by an anti-KIR2DL1 antibody conjugated to allophycocyanin (R&D Systems) and also high KIR2DL2/DL3 expression as detected by an anti-KIR2DL2/DL3 antibody conjugated to phycoerythrin (BD Bioscience). FIG. 10 shows results obtained with scFv-linker(20) AEKEDL and scFv-EEKKMP, with substantial down regulation of the targeted KIRs.

To downregulate NKG2A, an scFv reacting with NKG2A was used to suppress its expression in NK cells. The scFv sequence, which was derived from published European Patent Application No. EP2247619 A1 by Spee et al. was linked to the CD8 signal peptide and the ER retention sequences as illustrated in FIG. 2. Using the MSCV retroviral vector, the constructs were transduced in NK cells expanded from human peripheral blood, which had high NKG2A expression as detected by an anti-NKG2A antibody conjugated to phycoerythrin (Beckman Coulter). FIG. 11 shows substantial downregulation of NKG2A obtained with scFv-EEKKMP.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. An engineered immune cell comprising
a first polynucleotide sequence encoding an immune activating receptor, wherein the immune activating receptor comprises a first binding domain and a signaling domain; and
a second polynucleotide sequence encoding a target-binding molecule linked to a localizing domain, wherein the target-binding molecule linked to a localizing domain comprises a second binding domain and a localizing domain comprising an ER retention sequence, a Golgi retention sequence, or a PEST sequence.

2. The engineered immune cell of claim 1, wherein the first polynucleotide sequence and the second polynucleotide sequence are on the same polynucleotide molecule.

3. The engineered immune cell of claim 2, further comprising an internal ribosomal entry site or a 2A peptide-coding region site between the first polynucleotide sequence encoding the immune activating receptor and the second polynucleotide sequence encoding the target-binding molecule linked to a localizing domain.

4. The engineered immune cell of claim 1, wherein the first polynucleotide sequence and the second polynucleotide sequence are on different polynucleotide molecules.

5. The engineered immune cell of claim 1, wherein the localizing domain comprises an ER retention sequence.

6. The engineered immune cell of claim 5, wherein the ER retention sequence comprises a KDEL amino acid sequence.

7. The engineered immune cell of claim 5, wherein the ER retention sequence comprises a KKXX amino acid sequence, wherein X represents any amino acid.

8. The engineered immune cell of claim 1, wherein the localizing domain comprises a Golgi retention sequence.

9. The engineered immune cell of claim 8, wherein the Golgi retention sequence comprises a YQRL amino acid sequence.

10. The engineered immune cell of claim 1, wherein the localizing domain comprises a PEST sequence.

11. The engineered immune cell of claim 1, wherein the engineered immune cell is a T cell.

12. The engineered immune cell of claim 1, wherein the engineered immune cell is a natural killer (NK) cell.

13. The engineered immune cell of claim 1, wherein the first or the second binding domain comprises a single chain binding domain.

14. The engineered immune cell of claim 13, wherein the single chain binding domain is a single chain variable fragment (scFv).

15. The engineered immune cell of claim 1, wherein the target-binding molecule linked to a localizing domain further comprises a transmembrane domain.

16. The engineered immune cell of claim 15, wherein the transmembrane domain is a CD8 transmembrane domain.

17. The engineered immune cell of claim 1, wherein the immune activating receptor is a chimeric antigen receptor (CAR).

18. The engineered immune cell of claim 1, wherein the second binding domain is identical to the first binding domain.

19. The engineered immune cell of claim 1, wherein the second binding domain and the first binding domain are different.

20. The engineered immune cell of claim 1, wherein the signaling domain comprises a CD3ζ, a Fc, a DAP10, or a DAP12 domain.

21. The engineered immune cell of claim 1, wherein the immune activating receptor further comprises a co-stimulatory domain.

22. The engineered immune cell of claim 21, wherein the co-stimulatory domain comprises a 4-1BB domain, a CD28 domain, a CD28_{LL->GG} variant domain, an OX40 domain, an ICOS domain, a CD27 domain, a GITR domain, a HVEM domain, a TIM1 domain, a LFA1 domain, or a CD2 domain.

23. A pharmaceutical composition comprising an engineered immune cell of claim 1, and a pharmaceutically acceptable carrier.


SEQUENCE LISTING (64 sequences)

SEQ ID NO: 1 - protein, 10 aa, synthetic construct
  REGION 1..10: note = Myc tag
EQKLISEEDL

SEQ ID NO: 2 - DNA, 40 bp, synthetic construct
  misc_feature 1..40: note = Sense primer
  source 1..40: mol_type = other DNA
atatatgaattcggcttccaccatggccttaccagtgacc

SEQ ID NO: 3 - DNA, 50 bp, synthetic construct
  misc_feature 1..50: note = Reverse primer
  source 1..50: mol_type = other DNA
cagatcttcttcagaaataagtttttgttcggctgaggagactgtgagag

SEQ ID NO: 4 - protein, 4 aa, synthetic construct
  REGION 1..4: note = KDEL coding sequence
KDEL

SEQ ID NO: 5 - DNA, 40 bp, synthetic construct; identical to SEQ ID NO: 2
  misc_feature 1..40: note = Sense primer
  source 1..40: mol_type = other DNA

SEQ ID NO: 6 - DNA, 48 bp, synthetic construct
  misc_feature 1..48: note = Reverse primer
  source 1..48: mol_type = other DNA
tatatactcgagttacaactcgtccttcagatcttcttcagaaataag

SEQ ID NO: 7 - protein, 40 aa, synthetic construct
  REGION 1..40: note = PEST motif
SHGFPPEVEEQDDGTLPMSCAQESGMDRHPAACASARINV

SEQ ID NO: 8 - protein, 5 aa, synthetic construct
  REGION 1..5: note = Hydrophilic peptide
GGGGS

SEQ ID NO: 9-10 - skipped (2 entries)

SEQ ID NO: 11 - protein, 4 aa, synthetic construct
  REGION 1..4: note = ER or Golgi retention sequence
YQRL

SEQ ID NO: 12 - protein, 120 aa, synthetic construct
  REGION 1..120: note = Variable heavy region for scFv targeting CD3
EVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSA

SEQ ID NO: 13 - protein, 107 aa, synthetic construct
  REGION 1..107: note = Variable light region for scFv targeting CD3
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 14 - DNA, 360 bp, synthetic construct
  misc_feature 1..360: note = Variable heavy region for scFv targeting CD3
  source 1..360: mol_type = other DNA
gaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagcc

SEQ ID NO: 15 - DNA, 321 bp, synthetic construct
  misc_feature 1..321: note = Variable light region for scFv targeting CD3
  source 1..321: mol_type = other DNA
caaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaaccgg

SEQ ID NO: 16 - protein, 117 aa, synthetic construct
  REGION 1..117: note = Variable heavy region for scFv targeting CD7(TH69)
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSS

SEQ ID NO: 17 - protein, 112 aa, synthetic construct
  REGION 1..112: note = Variable light region for scFv targeting CD7(TH69)
AAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKR

SEQ ID NO: 18 - DNA, 351 bp, synthetic construct
  misc_feature 1..351: note = Variable heavy region for scFv targeting CD7(TH69)
  source 1..351: mol_type = other DNA
gaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagc

SEQ ID NO: 19 - DNA, 336 bp, synthetic construct
  misc_feature 1..336: note = Variable light region for scFv targeting CD7(TH69)
  source 1..336: mol_type = other DNA
gccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaagg

SEQ ID NO: 20 - protein, 123 aa, synthetic construct
  REGION 1..123: note = Variable heavy region for scFv targeting CD7(3a1f)
QVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSS

SEQ ID NO: 21 - protein, 108 aa, synthetic construct
  REGION 1..108: note = Variable light region for scFv targeting CD7(3a1f)
DIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKR

SEQ ID NO: 22 - DNA, 369 bp, synthetic construct
  misc_feature 1..369: note = Variable heavy region for scFv targeting CD7(3a1f)
  source 1..369: mol_type = other DNA
caggtccagctgcaggagtcaggggcagagctggtgaaacccggagccagtgtcaaactgtcctgtaaggccagcggctatactttcaccagctactggatgcactgggtgaaacagaggccaggacagggcctggagtggatcggcaagattaaccccagcaatgggcgcaccaactacaacgaaaagtttaaatccaaggctacactgactgtggacaagagctcctctaccgcatacatgcagctgagttcactgacatctgaagatagtgccgtgtactattgcgccagaggcggggtctactatgacctgtactattacgcactggattattgggggcagggaaccacagtgactgtcagctcc

SEQ ID NO: 23 - DNA, 324 bp, synthetic construct
  misc_feature 1..324: note = Variable light region for scFv targeting CD7(3a1f)
  source 1..324: mol_type = other DNA
gacatcgagctgacacagtctccagccactctgagcgtgacccctggcgattctgtcagtctgtcatgtagagctagccagtccatctctaacaatctgcactggtaccagcagaaatcacatgaaagccctcggctgctgattaagagtgcttcacagagcatctccgggattccaagcagattctctggcagtgggtcaggaaccgactttacactgtccattaactctgtggagaccgaagatttcggcatgtatttttgccagcagagcaattcctggccttacacattcggaggcgggactaaactggagattaagagg

SEQ ID NO: 24 - protein, 120 aa, synthetic construct
  REGION 1..120: note = Variable heavy region for scFv targeting CD45
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVS

SEQ ID NO: 25 - protein, 111 aa, synthetic construct
  REGION 1..111: note = Variable light region for scFv targeting CD45
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 26 - DNA, 360 bp, synthetic construct
  misc_feature 1..360: note = Variable heavy region for scFv targeting CD45
  source 1..360: mol_type = other DNA
caggtgcagctggtcgagtctggaggaggactggtgcagcctggaggaagtctgaagctgtcatgtgcagccagcgggttcgacttttctcgatactggatgagttgggtgcggcaggcaccaggaaaaggactggaatggatcggcgagattaacccaactagctccaccatcaatttcacacccagcctgaaggacaaagtgtttatttccagagataacgccaagaatactctgtatctgcagatgtccaaagtcaggtctgaagataccgccctgtactattgtgctcggggcaactactatagatacggggacgctatggattattgggggcagggaactagcgtgaccgtgagt

SEQ ID NO: 27 - DNA, 333 bp, synthetic construct
  misc_feature 1..333: note = Variable light region for scFv targeting CD45
  source 1..333: mol_type = other DNA
gacattgtgctgacccagtcccctgcttcactggcagtgagcctgggacagagggcaaccatcagctgccgagcctctaagagtgtctcaacaagcggatactcctatctgcactggtaccagcagaagccaggacagccacctaaactgctgatctatctggcttccaacctggaatctggagtgcctgcacgcttctccggatctggaagtggaaccgactttacactgaatattcacccagtcgaggaagaggatgccgctacctactattgccagcacagccgggagctgcccttcacatttggcagcgggactaagctggagatcaag

SEQ ID NO: 28 - protein, 119 aa, synthetic construct
  REGION 1..119: note = Variable heavy region for scFv targeting B2MG
EVQLQQSGAELVKPGASVKLSCTPSGFNVKDTYIHWVKQRPKQGLEWIGRIDPSDGDIKYDPKFQGKATITADTSSNTVSLQLSSLTSEDTAVYYCARWFGDYGAMNYWGQGTSVTVSS

SEQ ID NO: 29 - protein, 110 aa, synthetic construct
  REGION 1..110: note = Variable light region for scFv targeting B2MG
DIQMTQSPASQSASLGESVTITCLASQTIGTWLAWYQQKPGKSPQLLIYAATSLADGVPSRFSGSGSGTKFSLKIRTLQAEDFVSYYCQQLYSKPYTFGGGTKLEIKRAD

SEQ ID NO: 30 - DNA, 357 bp, synthetic construct
  misc_feature 1..357: note = Variable heavy region for scFv targeting B2MG
  source 1..357: mol_type = other DNA
gaggtgcagctgcagcagagcggagcagaactggtgaaacctggagccagcgtcaagctgtcctgtactccatctggcttcaacgtgaaggacacatacattcactgggtcaagcagcggcccaaacagggactggagtggatcggcagaattgacccatccgacggcgatatcaagtatgatcccaaattccaggggaaggctactattaccgcagataccagctccaacacagtgagtctgcagctgtctagtctgactagcgaagacaccgccgtctactattgtgctagatggtttggcgattacggggccatgaattattgggggcagggaaccagcgtcaccgtgtccagc

SEQ ID NO: 31 - DNA, 330 bp, synthetic construct
  misc_feature 1..330: note = Variable light region for scFv targeting B2MG
  source 1..330: mol_type = other DNA
gatattcagatgacccagtcccctgcatcacagagcgcctccctgggcgagtcagtgaccatcacatgcctggctagccagacaattggcacttggctggcatggtaccagcagaagcccggcaaatcccctcagctgctgatctatgcagctacctctctggcagacggagtgcccagtaggttctctgggagtggatcaggcaccaagttttctctgaaaattcgcacactgcaggctgaggatttcgtctcctactattgccagcagctgtactctaaaccttatacatttggcgggggaactaagctggaaatcaaacgagcagac

SEQ ID NO: 32 - protein, 119 aa, synthetic construct
  REGION 1..119: note = Variable heavy region for scFv targeting NKG2A
EVQLVESGGGLVKPGGSLKLSCAASGFTFSSYAMSWVRQSPEKRLEWVAEISSGGSYTYYPDTVTGRFTISRDNAKNTLYLEISSLRSEDTAMYYCTRHGDYPRFFDVWGAGTTVTVSS

SEQ ID NO: 33 - protein, 107 aa, synthetic construct
  REGION 1..107: note = Variable light region for scFv targeting NKG2A
QIVLTQSPALMSASPGEKVTMTCSASSSVSYIYWYQQKPRSSPKPWIYLTSNLASGVPARFSGSGSGTSYSLTISSMEAEDAATYYCQQWSGNPYTFGGGTKLEIKR

SEQ ID NO: 34 - DNA, 357 bp, synthetic construct
  misc_feature 1..357: note = Variable heavy region for scFv targeting NKG2A
  source 1..357: mol_type = other DNA
gaggtgcagctggtggagagcggaggaggactggtgaagccaggaggaagcctgaagctgtcctgtgccgcctctggcttcacattttcctcttatgcaatgagctgggtgcggcagtccccagagaagagactggagtgggtggcagagatcagctccggaggatcctacacctactatcctgacacagtgaccggccggttcacaatctctagagataacgccaagaataccctgtatctggagatctctagcctgagatccgaggatacagccatgtactattgcaccaggcacggcgactacccacgcttctttgacgtgtggggagcaggaaccacagtgaccgtgtcctct

SEQ ID NO: 35 - DNA, 321 bp, synthetic construct
  misc_feature 1..321: note = Variable light region for scFv targeting NKG2A
  source 1..321: mol_type = other DNA
cagattgtcctgacccagtctccagccctgatgagcgcctcccctggcgagaaggtgacaatgacctgctctgccagctcctctgtgagctacatctattggtaccagcagaagcctcggagctccccaaagccctggatctatctgacatccaacctggcctctggcgtgccagccagattctctggcagcggctccggcacatcttacagcctgaccatctctagcatggaggccgaggacgccgccacctactattgccagcagtggtccggcaatccatatacatttggcggcggcaccaagctggagatcaagagg

SEQ ID NO: 36 - protein, 122 aa, synthetic construct
  REGION 1..122: note = Variable heavy region for scFv targeting KIR2DL1and 2/3
QVQLVQSGAEVKKPGSSVKVSCKASGGTFSFYAISWVRQAPGQGLEWMGGFIPIFGAANYAQKFQGRVTITADESTSTAYMELSSLRSDDTAVYYCARPSGSYYYDYDMDVWGQGTTVTVSS

SEQ ID NO: 37 - protein, 109 aa, synthetic construct
  REGION 1..109: note = Variable light region for scFv targeting KIR2DL1and 2/3
EIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRT

SEQ ID NO: 38 - DNA, 369 bp, synthetic construct
  misc_feature 1..369: note = Variable heavy region for scFv targeting KIR2DL1and 2/3
  source 1..369: mol_type = other DNA
caggtccagctggtgcagtctggagctgaagtgaagaaaccagggagctccgtcaaggtgtcatgcaaagcaagcggcgggactttctccttttatgcaatctcttgggtgagacaggcacctggacagggactggagtggatgggaggcttcatcccaatttttggagccgctaactatgcccagaagttccagggcagggtgaccatcacagctgatgagtctactagtaccgcatacatggaactgtctagtctgaggagcgacgataccgccgtgtactattgtgctcgcattccatcaggcagctactattacgactatgatatggacgtgtggggccaggggaccacagtcaccgtgagcagc

SEQ ID NO: 39 - DNA, 327 bp, synthetic construct
  misc_feature 1..327: note = Variable light region for scFv targeting KIR2DL1and 2/3
  source 1..327: mol_type = other DNA
gagatcgtgctgacccagtctcctgtcacactgagtctgtcaccaggggaacgggctacactgtcttgcagagcaagccagtccgtgagctcctacctggcctggtatcagcagaagccaggccaggctcccaggctgctgatctacgatgcaagcaacagggccactgggattcccgcccgcttctctggcagtgggtcaggaaccgactttactctgaccatttctagtctggagcctgaagatttcgccgtgtactattgccagcagcgatccaattggatgtatacttttggccaggggaccaagctggagatcaaacggaca

SEQ ID NO: 40 - protein, 21 aa, synthetic construct
  REGION 1..21: note = CD8 signal peptide
MALPVTALLLPLALLLHAARP

SEQ ID NO: 41 - protein, 20 aa, synthetic construct
  REGION 1..20: note = Variable heavy-variable light linker
GGGGSGGGGSGGGGSGGGGS

SEQ ID NO: 42 - protein, 70 aa, synthetic construct
  REGION 1..70: note = CD8 hinge and transmembrane
KPTTTPAPRPPTPAPTIASQPLSLRPEACRPAAGGAVHTRGLDFACDIYIWAPLAGTCGVLLLSLVITLY

SEQ ID NO: 43 - protein, 15 aa, synthetic construct
  REGION 1..15: note = Variable heavy-variable light linker
GGGGSGGGGSGGGGS

SEQ ID NO: 44 - DNA, 63 bp, synthetic construct
  misc_feature 1..63: note = CD8 signal peptide
  source 1..63: mol_type = other DNA
atggccttaccagtgaccgccttgctcctgccgctggccttgctgctccacgccgccaggccg

SEQ ID NO: 45 - DNA, 63 bp, synthetic construct
  misc_feature 1..63: note = CD8 signal peptide
  source 1..63: mol_type = other DNA
atggctctgcctgtgaccgcactgctgctgcccctggctctgctgctgcacgccgcaagacct

SEQ ID NO: 46 - DNA, 63 bp, synthetic construct
  misc_feature 1..63: note = CD8 signal peptide
  source 1..63: mol_type = other DNA
atggctctgcccgtcaccgctctgctgctgcctctggctctgctgctgcacgctgctcgacca

SEQ ID NO: 47 - DNA, 63 bp, synthetic construct
  misc_feature 1..63: note = CD8 signal peptide
  source 1..63: mol_type = other DNA
atggctctgcccgtgaccgctctgctgctgcctctggctctgctgctgcatgctgctcgacct

SEQ ID NO: 48 - DNA, 63 bp, synthetic construct
  misc_feature 1..63: note = CD8 signal peptide
  source 1..63: mol_type = other DNA
atggccctgcccgtcaccgccctgctgctgcccctggctctgctgctgcacgccgcaagaccc

SEQ ID NO: 49 - DNA, 63 bp, synthetic construct
  misc_feature 1..63: note = CD8 signal peptide
  source 1..63: mol_type = other DNA
atggctctgcccgtgaccgccctgctgctgcctctggctctgctgctgcacgctgcccgccca

SEQ ID NO: 50 - protein, 68 aa, synthetic construct
  REGION 1..68: note = CD8 hinge and transmembrane
TTTPAPRPPTPAPTIASQPLSLRPEACRPAAGGAVHTRGLDFACDIYIWAPLAGTCGVLLLSLVITLY

SEQ ID NO: 51 - DNA, 60 bp, synthetic construct
  misc_feature 1..60: note = Variable heavy-variable light linker
  source 1..60: mol_type = other DNA
ggtggtggtggttctggtggtggtggttctggcggcggcggctccggtggtggtggatcc

SEQ ID NO: 52 - DNA, 60 bp, synthetic construct
  misc_feature 1..60: note = Variable heavy-variable light linker
  source 1..60: mol_type = other DNA
ggaggaggaggaagcggaggaggaggatccggaggcgggggatctggaggaggaggaagt

SEQ ID NO: 53 - DNA, 45 bp, synthetic construct
  misc_feature 1..45: note = Variable heavy-variable light linker
  source 1..45: mol_type = other DNA
ggaggaggaggatccggcggaggaggctctgggggaggcgggagt

SEQ ID NO: 54 - DNA, 60 bp, synthetic construct
  misc_feature 1..60: note = Variable heavy-variable light linker
  source 1..60: mol_type = other DNA
ggaggaggaggaagtggaggaggaggatcaggaggcgggggaagcggcgggggaggctcc

SEQ ID NO: 55 - DNA, 60 bp, synthetic construct
  misc_feature 1..60: note = Variable heavy-variable light linker
  source 1..60: mol_type = other DNA
ggaggaggaggaagtggaggaggagggtcaggaggcgggggaagcggcgggggaggatcc

SEQ ID NO: 56 - DNA, 60 bp, synthetic construct
  misc_feature 1..60: note = Variable heavy-variable light linker
  source 1..60: mol_type = other DNA
ggaggaggaggatctggaggaggaggcagcggcggcggcggctccggcggcggcggctct

SEQ ID NO: 57 - DNA, 210 bp, synthetic construct
  misc_feature 1..210: note = CD8 hinge and transmembrane
  source 1..210: mol_type = other DNA
aagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttac

SEQ ID NO: 58 - DNA, 204 bp, synthetic construct
  misc_feature 1..204: note = CD8 hinge and transmembrane
  source 1..204: mol_type = other DNA
accactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactttac

SEQ ID NO: 59 - DNA, 204 bp, synthetic construct
  misc_feature 1..204: note = CD8 hinge and transmembrane
  source 1..204: mol_type = other DNA
actaccacaccagctccaagaccacctacccctgcaccaacaattgctagtcagccactgtcactgagaccagaagcatgtaggcctgcagctggaggagctgtgcacaccagaggcctggactttgcctgcgatatctacatttgggctcctctggcaggaacctgtggcgtgctgctgctgtctctggtcatcacactttac

SEQ ID NO: 60 - DNA, 210 bp, synthetic construct
  misc_feature 1..210: note = CD8 hinge and transmembrane
  source 1..210: mol_type = other DNA
aagccaaccacaacccctgcaccaaggccacctacaccagcacctaccatcgcaagccagccactgtccctgaggccagaggcatgtaggcctgcagcaggaggcgccgtgcacacacgcggcctggactttgcctgcgatatctacatctgggcaccactggcaggaacctgtggcgtgctgctgctgagcctggtgattaccctgtat

SEQ ID NO: 61 - DNA, 30 bp, synthetic construct
  misc_feature 1..30: note = Variable heavy-variable light linker
  source 1..30: mol_type = other DNA
ggtggtggcggcagtggtggcggtggctca

SEQ ID NO: 62 - protein, 10 aa, synthetic construct
  REGION 1..10: note = Variable heavy-variable light linker
GGGGSGGGGS

SEQ ID NO: 63 - DNA, 60 bp, synthetic construct
  misc_feature 1..60: note = Variable heavy-variable light linker
  source 1..60: mol_type = other DNA
ggtggtggcggcagtggtggcggtggctcaggcggtggtggctccggtggcggtggctct

SEQ ID NO: 64 - protein, 6 aa, synthetic construct
  REGION 1..6: note = Variable heavy-variable light linker
EEKKMP